(19) United States
(12) Patent Application Publication
(10) Pub. No.: US 2022/0313973 A1
(43) Pub. Date: Oct. 6, 2022

(54) Instrument Advancement Device Having an Indicator Marking

(71) Applicant: Becton, Dickinson and Company, Franklin Lakes, NJ (US)

(72) Inventors: Curtis H. Blanchard, Riverton, UT (US); Jonathan Karl Burkholz, Salt Lake City, UT (US); Weston F. Harding, Lehi, UT (US); Megan Scherich, Salt Lake City, UT (US); Bart D. Peterson, Farmington, UT (US); John M. Lackey, West Valley City, UT (US); Thomas A. Solosko, Draper, UT (US)

(21) Appl. No.: 17/710,015

(22) Filed: Mar. 31, 2022

Related U.S. Application Data
(60) Provisional application No. 63/170,391, filed on Apr. 2, 2021.
(60) Provisional application No. 63/309,918, filed on Feb. 14, 2022.

(51) Int. Cl. A61M 39/02; A61M 25/00

(52) U.S. Cl. CPC A61M 39/0247; A61M 25/0097; A61M 2039/1077

(57) ABSTRACT

An instrument advancement device may include an instrument and an advancement feature configured to advance the instrument distally through the catheter assembly. The instrument advancement device may include a first indicator marking, a second indicator marking, and a third indicator marking. In response to aligning the advancement feature with the first indicator marking, a distal tip of the instrument may be configured to align with a distal tip of a catheter assembly. In response to aligning the advancement feature with the second indicator marking, the distal tip of the instrument may be advanced a distance distal to the distal tip of the catheter assembly. In response to aligning the advancement feature with the third indicator marking, the distal tip of the instrument may be advanced another distance distal to the distal tip of the catheter assembly.

### CROSS-REFERENCE TO RELATED APPLICATION

The present application claims priority to U.S. Provisional Application Ser. No. 63/170,391, entitled “Instrument Advancement Device Having an Indicator Marking”, filed Apr. 2, 2021, and U.S. Provisional Application Ser. No. 63/309,918, entitled “Instrument Advancement Device Having an Indicator Marking”, filed Feb. 14, 2022, the entire disclosures of both of which are hereby incorporated by reference in their entireties.

### BACKGROUND

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of intravenous (IV) catheter device includes a catheter that is over-the-needle. As its name implies, the catheter that is over-the-needle may be mounted over an introducer needle having a sharp distal tip. The IV catheter device may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increases. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to instrument advancement devices to facilitate advancement and/or retraction of an instrument within a catheter assembly, as well as related systems and methods. According to a first set of embodiments, a method of manufacture may include providing a first catheter assembly. In some embodiments, the first catheter assembly may include a first catheter adapter, which may include a distal end, a proximal end, and a lumen extending through the distal end of the first catheter adapter and the proximal end of the first catheter adapter. In some embodiments, the first catheter assembly may include a first catheter extending from the distal end of the first catheter adapter. In some embodiments, the first catheter may include a first length. In some embodiments, the first catheter assembly may include a first indicator marking corresponding to the first length.

In some embodiments, the method of manufacture may include providing a second catheter assembly. In some embodiments, the second catheter assembly may include a second catheter adapter, which may include a distal end, a proximal end, and a lumen extending through the distal end of the second catheter adapter and the proximal end of the second catheter adapter. In some embodiments, the second catheter assembly may include a second catheter extending from the distal end of the second catheter adapter. In some embodiments, the second catheter may include a second length. In some embodiments, the second catheter assembly may include a second indicator marking corresponding to the second length.

In some embodiments, the method of manufacture may include providing an instrument advancement device configured to couple to the first catheter assembly and the second catheter assembly. In some embodiments, the instrument advancement device may be configured to advance an instrument distally into the first catheter assembly and the second catheter assembly. In some embodiments, the instrument advancement device may include a first other indicator marking matching the first indicator marking and a second other indicator marking matching the second indicator marking. In some embodiments, a particular instrument advancement device may include any number of other indicator markings.

In some embodiments, the first catheter assembly may include a first connector coupled to the first catheter adapter. In some embodiments, an outer surface of the first connector may include the first indicator marking. In some embodiments, the second catheter assembly may include a second connector coupled to the second catheter adapter. In some embodiments, an outer surface of the second connector may include the second indicator marking.

In some embodiments, the instrument advancement device may include an advancement feature. In some embodiments, in response to aligning the advancement feature with the first other indicator marking, a distal tip of the instrument may be configured to align with a distal tip of the first catheter. In some embodiments, in response to aligning the advancement feature with the second other indicator marking, the distal tip of the instrument may be configured to align with the distal tip of the second catheter.

In some embodiments, the method of manufacture may include providing a third catheter assembly. In some embodiments, the third catheter assembly may include a third catheter adapter, which may include a distal end, a proximal end, and a lumen extending through the distal end of the third catheter adapter and the proximal end of the third catheter adapter. In some embodiments, the third catheter assembly may include a third catheter extending from the distal end of the third catheter adapter. In some embodiments, the third catheter may include a third length. In some embodiments, the third catheter assembly may include a third indicator marking corresponding to the third length. In some embodiments, the instrument advancement device includes a third other indicator marking matching the third indicator marking. In some embodiments, in response to aligning the advancement feature with the third other indicator marking, the distal tip of the instrument may be configured to align with a distal tip of the third catheter.

In some embodiments, the second other indicator marking and the third other indicator marking may include a same color, and the first other indicator marking may include a different color than the same color. In some embodiments, the first indicator marking may include a same color and/or a same printed length as the first other indicator marking. In some embodiments, the second indicator marking may include a same other color and/or a same other printed length as the second other indicator marking. In some embodiments, the third indicator marking may include a same other color and/or a same other printed length as the third other indicator marking.

In some embodiments, the instrument advancement device may include a housing, a tube extending through the housing, a wedge disposed within the housing, and a pair of opposing pinch members configured to pinch the tube. In some embodiments, the pair of opposing pinch members may be disposed within the housing and configured to move along the tube with the housing. In some embodiments, the instrument may be disposed within the tube. In some embodiments, in response to moving the housing distally along the tube, the pair of opposing pinch members may push the wedge distally and the instrument may be configured to advance distally into the first catheter assembly and the second catheter assembly. In some embodiments, the first other indicator marking and the second other indicator marking may be disposed on the tube.

In some embodiments, the first other indicator marking may include a first line having a proximal end and a distal end. In some embodiments, the second other indicator marking may include a second line having a proximal end and a distal end. In some embodiments, in response to aligning the housing with the proximal end of the first line, a distal tip of the instrument may be configured to align with a distal tip of the first catheter. In some embodiments, in response to aligning the housing with the proximal end of the second line, the distal tip of the instrument may be configured to align with the distal tip of the second catheter. In some embodiments, in response to aligning the housing with the distal end of the first line, the distal tip of the instrument may be configured to extend distal to the distal tip of the first catheter. In some embodiments, in response to aligning the housing with the distal end of the second line, the distal tip of the instrument may be configured to extend beyond the distal tip of the second catheter.

In some embodiments, the instrument advancement device may include a housing, which may include a proximal end, a distal end, and a slot. In some embodiments, the instrument advancement device may include an advancement feature extending through the slot and configured to move linearly along the slot between a retracted position and an advanced position. In some embodiments, the instrument may include a first end and a second end, which may include the distal tip. In some embodiments, in response to movement of the advancement feature from the retracted position to the advanced position, the second end of the instrument may be advanced beyond the distal end of the housing. In some embodiments, the first other indicator marking and the second other indicator marking may be disposed on the housing.

In some embodiments, the instrument advancement device may include a housing, which may include a distal end and a proximal end. In some embodiments, the distal end of the housing may be configured to couple to an intravenous catheter device. In some embodiments, the instrument may be disposed within the housing. In some embodiments, the instrument advancement device may include an advancement wheel. In some embodiments, the advancement wheel may extend out from the housing. In some embodiments, in response to the advancement wheel being rotated, the instrument may be advanced through the distal end of the housing. In some embodiments, the first indicator marking, and the second indicator marking may be disposed on the housing.

According to a second set of embodiments, in some embodiments, an instrument advancement device may include the instrument, which may include an indicator marking. In some embodiments, the instrument advancement device may include a window. In some embodiments, in response to movement of the indicator marking to a position within the window, a distal tip of the instrument may be advanced distally a distance. In some embodiments, the instrument advancement device may include another window aligned with and distal to the window. In some embodiments, in response to the indicator marking being within the window, the distal tip of the instrument may be advanced distally another distance. In some embodiments, the other distance may be greater than the distance.

According to a third set of embodiments, an instrument advancement device may include a housing, a tube extending through the housing, a wedge disposed within the housing, and a pair of opposing pinch members configured to pinch the tube. In some embodiments, the pair of opposing pinch members may be disposed within the housing and configured to move along the tube with the housing. In some embodiments, the window may be disposed within the tube. In some embodiments, the instrument may be disposed within the tube. In some embodiments, in response to moving the housing distally along the tube, the pair of opposing pinch members may push the wedge distally, the indicator marking may be disposed within the window, and the distal tip of the instrument is advanced distally a distance beyond the tube. In some embodiments, in response to moving the housing distally further along the tube the pair of opposing pinch members may push the wedge distally, the indicator marking may be disposed within the other window, and the distal tip of the instrument may be advanced distally another distance beyond the tube. In some embodiments, the other distance may be greater than the distance.

In some embodiments, the instrument advancement device may include a housing, which may include a distal end and a proximal end. In some embodiments, the distal end of the housing may be configured to couple to an intravenous catheter device. In some embodiments, the window may be disposed within the housing. In some embodiments, the instrument may be disposed within the housing. In some embodiments, the instrument advancement device may include an advancement wheel. In some embodiments, the advancement wheel may extend out from the housing. In some embodiments, in response to the advancement wheel being rotated, the indicator marking may be disposed within the window, and the distal tip of the instrument may be advanced distally through the distal end of the housing a distance.

According to a fourth set of embodiments, an instrument advancement device may include the instrument and the advancement feature configured to advance the instrument distally through the catheter assembly. In some embodiments, the instrument advancement device may include a first indicator marking, a second indicator marking, and a third indicator marking. In some embodiments, in response to aligning the advancement feature with the first indicator marking, a distal tip of the instrument may be configured to align with a distal tip of a catheter assembly. In some embodiments, in response to aligning the advancement feature with the second indicator marking, the distal tip of the instrument may be advanced a distance distal to the distal tip of the catheter assembly. In some embodiments, in response to aligning the advancement feature with the third indicator marking, the distal tip of the instrument may be advanced another distance distal to the distal tip of the catheter assembly.

In some embodiments, the first indicator marking may include a printed T or a printed word “Tip”. In some embodiments, the term “printed” may refer to an alphabetical descriptor or a numeric descriptor. In some embodiments, the second indicator may include a printed number corresponding to the distance. In some embodiments, the third indicator may include a printed number corresponding to the other distance.

In some embodiments, the instrument advancement device may include a housing, which may include a proximal end, a distal end, and a slot. In some embodiments, the instrument may be disposed within the housing. In some embodiments, the advancement feature may extend through the slot and may be configured to move linearly along the slot between a retracted position and an advanced position. In some embodiments, in response to movement of the advancement feature from the retracted position to the advanced position, the second end of the instrument may be advanced beyond the distal end of the housing. In some embodiments, the first other indicator marking, the second other indicator marking, and the third other indicator marking may be disposed on the housing.

In some embodiments, the instrument advancement device may include a housing, which may include a distal end and a proximal end. In some embodiments, the distal end of the housing may be configured to couple to an intravenous catheter device. In some embodiments, the instrument may be disposed within the housing. In some embodiments, the advancement feature may include an advancement wheel. In some embodiments, the advancement wheel may extend out from the housing. In some embodiments, in response to the advancement wheel being rotated, the instrument may be advanced through the distal end of the housing. In some embodiments, the first other indicator marking, the second other indicator marking, and the third other indicator marking may be disposed on the housing.

In some embodiments, the instrument advancement device may include a housing, a tube extending through the housing, a wedge disposed within the housing, and a pair of opposing pinch members configured to pinch the tube. In some embodiments, the pair of opposing pinch members are disposed within the housing and configured to move along the tube with the housing, wherein the instrument is disposed within the tube, wherein in response to moving the housing distally along the tube, the pair of opposing pinch members push the wedge distally and the instrument configured to advanced distally into the first catheter assembly and the second catheter assembly, wherein the first other indicator marking, the second other indicator marking, and the third other indicator marking may be disposed on the tube.

According to a fifth set of embodiments, an instrument advancement device may include a housing, which may include a distal end and a proximal end. In some embodiments, the distal end of the housing may be configured to couple to an intravenous catheter device. In some embodiments, an inner surface of the housing may include a housing stop member and an indicator marking. In some embodiments, the instrument advancement device may include a first wheel and/or an inner surface of the first wheel may include a first wheel stop. In some embodiments, the instrument advancement device may include a second wheel and/or the second wheel may include a tab. In some embodiments, in response to the instrument advancement device being disposed in a first configuration, the first wheel and the second wheel may be prevented from rotating in a first direction but are configured to rotate in a second direction opposite the first direction. In some embodiments, the first wheel may be configured to rotate more than one full turn in the second direction.

In some embodiments, the instrument advancement device may include a cover coupled to the second wheel and configured to rotate with the second wheel. In some embodiments, the cover may include a window. In some embodiments, when the instrument advancement device is in the first configuration, the cover may hide the indicator marking. In some embodiments, in response to rotation of the second wheel in the second direction from the first configuration, the indicator marking may be disposed in the window.

According to a sixth set of embodiments, a method of manufacture may include providing a first catheter assembly. In some embodiments, the first catheter assembly may include a first catheter adapter, which may include a distal end, a proximal end, a side port disposed between the distal end of the first catheter adapter and a proximal end of the first catheter adapter, and a lumen extending through the distal end of the first catheter adapter and the proximal end of the first catheter adapter.

In some embodiments, the first catheter assembly may include a first catheter extending from the distal end of the first catheter adapter. In some embodiments, the first catheter may include a first catheter length. In some embodiments, the first catheter assembly may include a first extension tube extending from the side port of the first catheter adapter. In some embodiments, the first extension tube may include a first extension tube length. In some embodiments, a first connector may be coupled to a proximal end of the first extension tube.

In some embodiments, the method of manufacture may include providing a second catheter assembly. In some embodiments, the second catheter assembly may include a second catheter adapter, which may include a distal end, a proximal end, a side port disposed between the distal end of the second catheter adapter and a proximal end of the second catheter adapter, and a lumen extending through the distal end of the second catheter adapter and the proximal end of the second catheter adapter.

In some embodiments, the second catheter assembly may include a second catheter extending from the distal end of the second catheter adapter. In some embodiments, the second catheter may include a second catheter length. In some embodiments, the second catheter assembly may include a second extension tube extending from the side port of the second catheter adapter. In some embodiments, the second extension tube may include a second extension tube length. In some embodiments, a second connector may be coupled to a proximal end of the second extension tube.

In some embodiments, the first catheter length may be less than the second catheter length. In some embodiments, the first extension tube may be longer than the second extension tube. In some embodiments, an entire length of the first catheter assembly from a distal tip of the first catheter to a proximal end of the first connector may be equal to an entire length of the second catheter assembly from a distal tip of the second catheter to a proximal end of the second connector. In some embodiments, the first connector may include a first connector length, and the second connector may include a second connector length. In some embodiments, the first connector length may be equal to the second connector length.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example catheter assembly, according to some embodiments;

FIG. 1B is a cross-sectional view of the catheter assembly of FIG. 1A, according to some embodiments;

FIG. 2A is an upper perspective view of another example catheter assembly, according to some embodiments;

FIG. 2B is a cross-sectional view of the catheter assembly of FIG. 2A, according to some embodiments;

FIGS. 3A-3L are upper perspective views of another example catheter assembly, according to some embodiments;

FIG. 4A is an upper perspective view of an example instrument advancement device, according to some embodiments;

FIG. 4B is an upper perspective view of another example instrument advancement device, according to some embodiments;

FIG. 4C is a cross-sectional view of an example distal end of the instrument advancement device of FIGS. 4A-4B, according to some embodiments;

FIG. 4D is a cross-sectional view of another example distal end of the instrument advancement device of FIGS. 4A-4B, according to some embodiments;

FIG. 4E is a cross-sectional view of a portion of the instrument advancement device of FIGS. 4A-4B, according to some embodiments;

FIG. 5A is an upper perspective view of another example instrument advancement device, according to some embodiments;

FIG. 5B is an upper perspective view of another example instrument advancement device, according to some embodiments;

FIG. 5C is a cross-sectional view of the instrument advancement device of FIG. 5B, according to some embodiments;

FIG. 5D is a cross-sectional view of the instrument advancement device of FIG. 5B along the line 5D-5D, according to some embodiments;

FIG. 5E is an enlarged view of a portion of FIG. 5D, according to some embodiments;

FIG. 5F is a cross-sectional view of the instrument advancement device of FIG. 5B along the line 5F-5F, according to some embodiments;

FIG. 6A is an upper perspective view of another example instrument advancement device, according to some embodiments;

FIG. 6B is a cross-sectional view of another example instrument advancement device, according to some embodiments;

FIG. 6C is an upper perspective view of an example instrument delivery mechanism, according to some embodiments;

FIG. 7 is an upper perspective view of another example instrument advancement device, according to some embodiments;

FIG. 8 is an upper perspective view of another example instrument advancement device, according to some embodiments;

FIG. 9 is an upper perspective view of another example instrument advancement device, according to some embodiments;

FIG. 10A is an upper perspective view of another example instrument advancement device, according to some embodiments;

FIG. 10B is an upper perspective view of another example instrument advancement device, according to some embodiments;

FIG. 11A is a cross-sectional side view of another example instrument advancement device, illustrating the instrument advancement device in a first configuration, according to some embodiments;

FIG. 11B is an upper perspective view of the instrument advancement device of FIG. 11A, illustrating an example cover in the first configuration, according to some embodiments;

FIG. 11C is a cross-sectional side view of the instrument advancement device of FIG. 11A, illustrating an example first wheel rotated independently from an example second wheel in a second direction from the first configuration, according to some embodiments;

FIG. 11D is a cross-sectional side view of the instrument advancement device of FIG. 11A, illustrating the first wheel rotated further in the second direction from the position of FIG. 11C, according to some embodiments;

FIG. 11E is a cross-sectional side view of the instrument advancement device of FIG. 11A, illustrating the first wheel and the second wheel rotated together in the second direction from the position of FIG. 11D, according to some embodiments;

FIG. 11F is an upper perspective view of the instrument advancement device of FIG. 11A, illustrating an example cover in the position of FIG. 11E, according to some embodiments;

FIG. 11G is a cross-sectional side view of another instrument advancement device, illustrating the instrument advancement device in a second configuration, according to some embodiments;

FIG. 12 is an upper perspective view of another example instrument advancement device, according to some embodiments;

FIG. 13 is an upper perspective view of another example instrument advancement device, according to some embodiments;

FIG. 14 is an upper perspective view of another example instrument advancement device, according to some embodiments;

FIG. 15 is an upper perspective view of another example instrument advancement device, according to some embodiments;

FIG. 16 is top view of another example catheter assembly, according to some embodiments; and

FIG. 17 is top view of another example catheter assembly, according to some embodiments.

### DESCRIPTION OF EMBODIMENTS

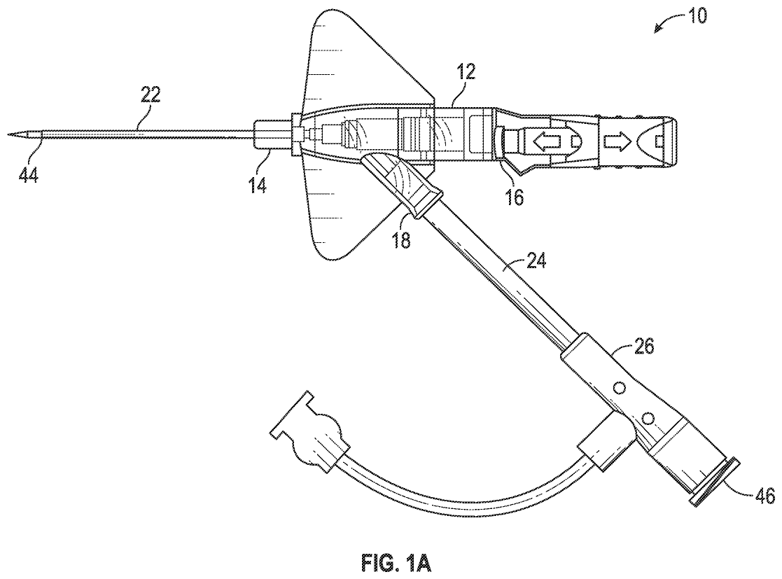

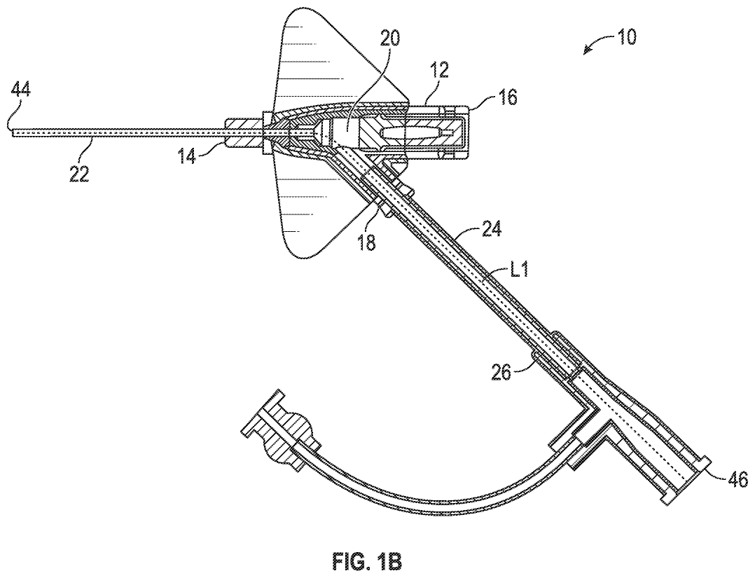

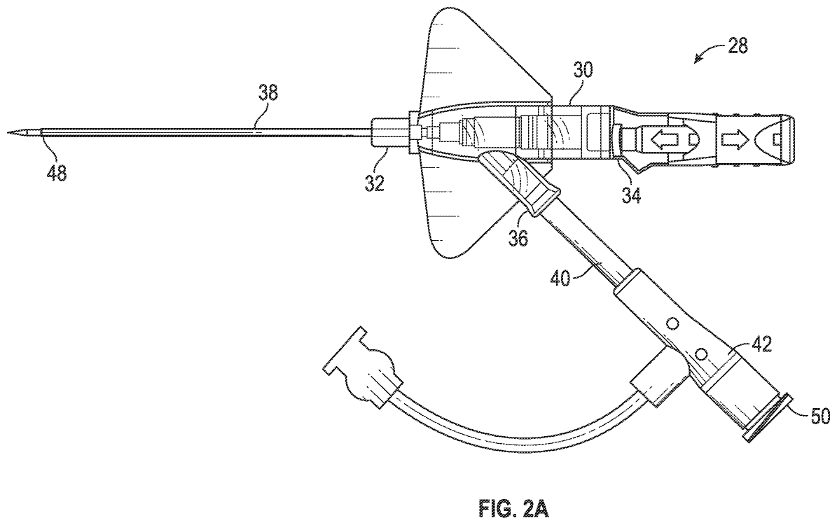

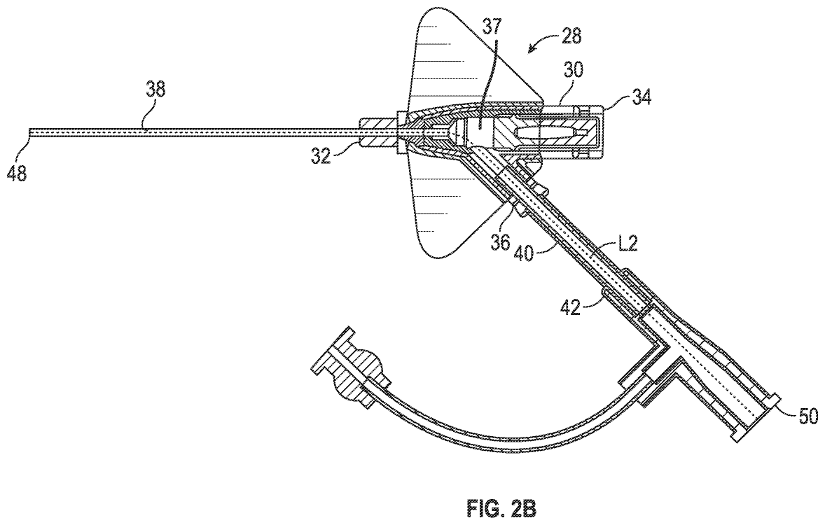

Referring now to FIGS. 1-2, in some embodiments, a first catheter assembly 10 may include a first catheter adapter 12, which may include a distal end 14 and a proximal end 16. In some embodiments, the first catheter adapter 12 may include a side port 18 disposed between the distal end 14 and a proximal end 16. In some embodiments, the first catheter adapter 12 may include a lumen 20 extending through the distal end 14 and the proximal end 16. FIGS. 1B and 2B illustrated a needle hub and introducer needle removed, according to some embodiments.

In some embodiments, the first catheter assembly 10 may include a first catheter 22 extending from the distal end 14. In some embodiments, the first catheter 22 may include a peripheral intravenous catheter, a midline catheter, or a peripherally-inserted central catheter. In some embodiments, the first catheter 22 may include a first catheter length. In some embodiments, the first catheter assembly 10 may include a first extension tube 24 extending from the side port

In some embodiments, the first extension tube 24 may include a first extension tube length. In some embodiments, a first connector 26 may be coupled to a proximal end of the first extension tube 24.

In some embodiments, a second catheter assembly 28 may include a second catheter adapter 30, which may include a distal end 32 and a proximal end 34. In some embodiments, the second catheter assembly 28 may be similar or identical to the first catheter assembly 10 in terms of one or more features and/or operation. In some embodiments, a side port 36 may be disposed between the distal end 32 and the proximal end 34. In some embodiments, a lumen 37 may extend through the distal end 32 and the proximal end 34.

In some embodiments, the second catheter assembly 28 may include a second catheter 38 extending from the distal end 32. In some embodiments, the second catheter 38 may include a peripheral intravenous catheter, a midline catheter, or a peripherally-inserted central catheter. In some embodiments, the second catheter 38 may include a second catheter length. In some embodiments, the second catheter assembly 28 may include a second extension tube 40 extending from the side port 36. In some embodiments, the second extension tube 40 may include a second extension tube length. In some embodiments, a second connector 42 may be coupled to a proximal end of the second extension tube 40.

In some embodiments, the first catheter length may be less than the second catheter length. In some embodiments, the first extension tube 24 may be longer than the second extension tube 40. In some embodiments, an entire length L1 of the first catheter assembly 10 from a distal tip 44 of the first catheter 22 to a proximal end 46 of the first connector 26 may be equal to an entire length L2 of the second catheter assembly 28 from a distal tip 48 of the second catheter 38 to a proximal end 50 of the second connector 42. In some embodiments, the first connector 26 may include a first connector length, and the second connector 42 may include a second connector length. In some embodiments, the first connector length may be equal to the second connector length.

In some embodiments, a clinician may desire to know when an instrument enters a vein or advances beyond a particular distal tip of a particular catheter assembly. In some embodiments, this is accomplished through manufacturing a set of catheter assemblies that have a same length from the distal tip of the catheter to the proximal end of the connector. For example, because the entire length L1 of the first catheter assembly 10 is equal to the entire length L2 of the second catheter assembly 28, advancement of the instrument within the first catheter assembly 10 and the second catheter assembly 28 may be predictable. In some embodiments, a method of manufacture may include providing the first catheter assembly 10 and the second catheter assembly 28, which may be part of a set or a kit.

In some embodiments, the proximal end 46 of the first connector 26 may be configured to couple to an instrument advancement device that includes the instrument. In some embodiments, the entire length L1 of the first catheter assembly 10 may include a path through which the instrument extends within the first catheter assembly 10. In some embodiments, the proximal end 50 of the second connector 42 may be configured to couple to the instrument advancement device. In some embodiments, the entire length L2 of the second catheter assembly 28 may include a path through which the instrument extends within the second catheter assembly 28.

In some embodiments, a configuration of the first catheter assembly 10 and/or the second catheter assembly 28 may vary. In some embodiments, the first catheter assembly 10 may include an integrated catheter assembly having the first extension tube 24 integrated with the side port 18. In other embodiments, the first catheter assembly 10 may include a straight catheter assembly or another suitable configuration. Similarly, in some embodiments, the second catheter assembly 28 may include an integrated catheter assembly having the second extension tube 40 integrated with the side port 18. In other embodiments, the second catheter assembly 28 may include a straight catheter assembly or another suitable configuration.

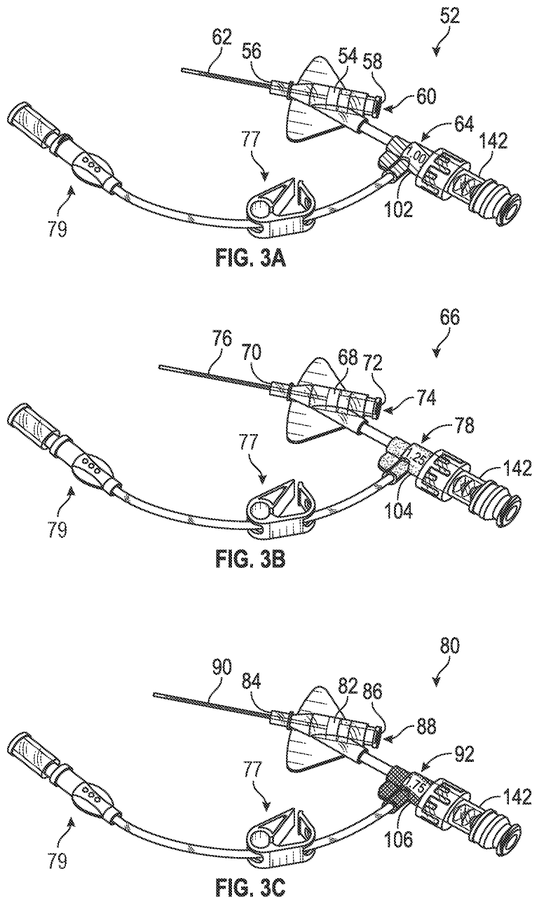

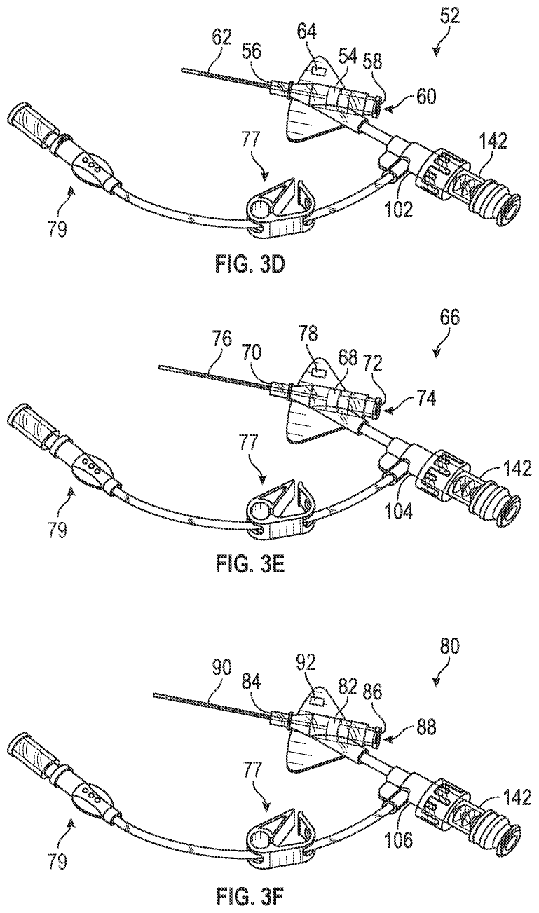

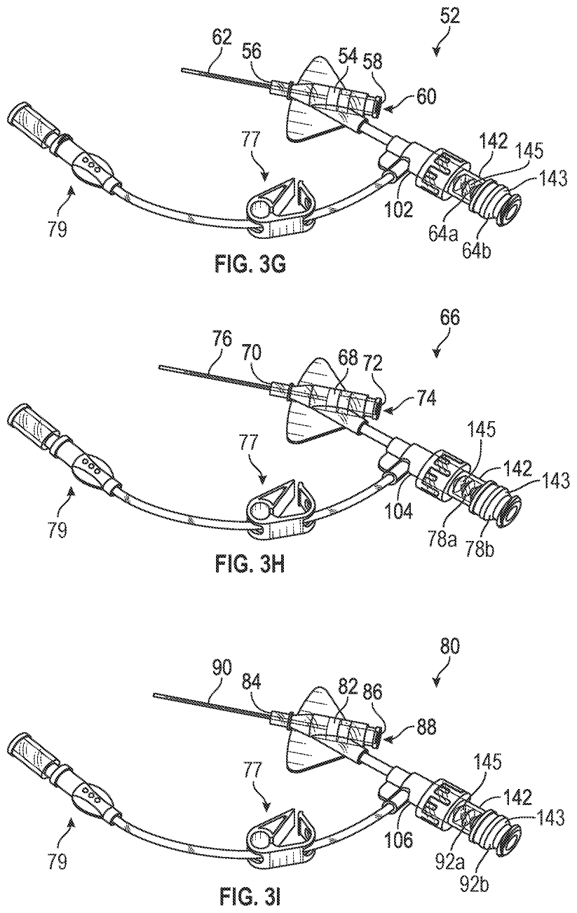

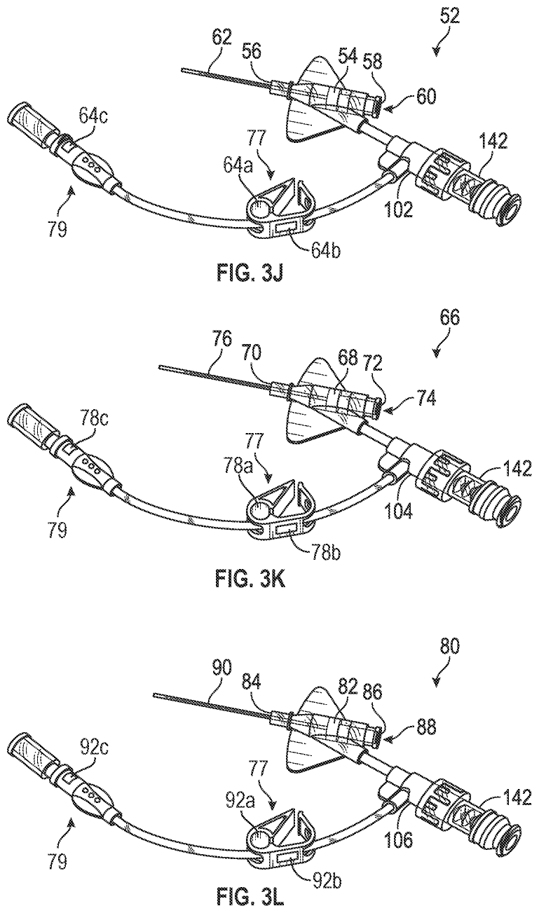

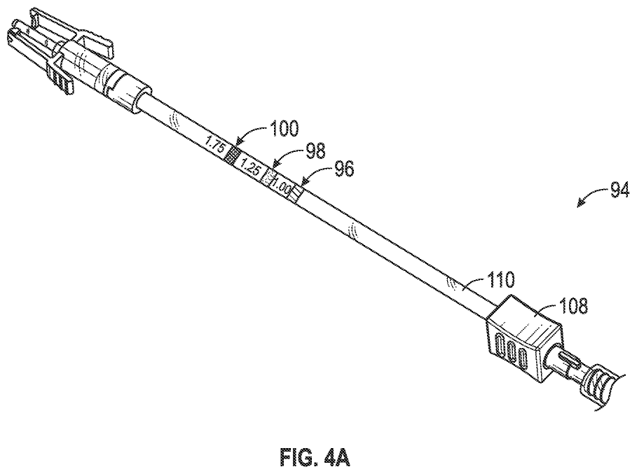

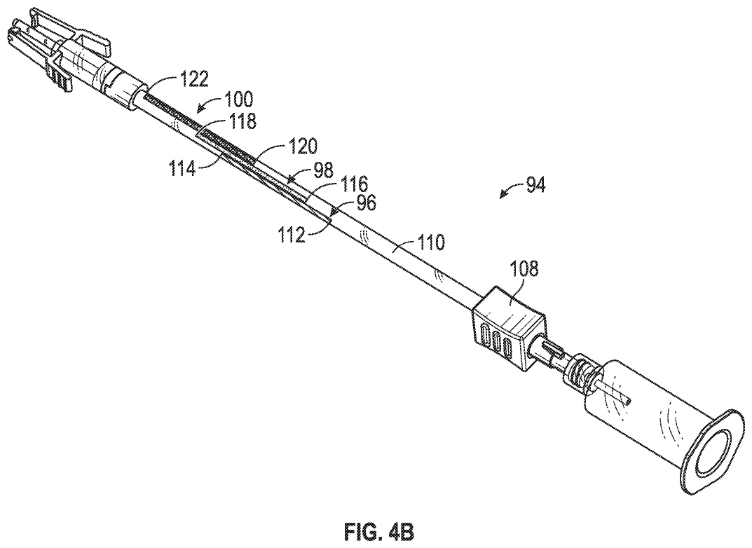

Referring now to FIGS. 3A-4A, in some embodiments, a first catheter assembly 52 may include a first catheter adapter 54, which may include a distal end 56, a proximal end 58, and a lumen 60 extending through the distal end 56 and the proximal end 58. In some embodiments, the first catheter assembly 52 may include a first catheter 62 extending from the distal end 56. In some embodiments, the first catheter 62 may include a first length. In some embodiments, the first catheter assembly 52 may include a first indicator marking 64 corresponding to the first length or indicating to the clinician the first length. As shown in the illustrated, non-limiting embodiments of FIGS. 3A-3L, first indicator marking 64 can be arranged on any number of components, and at any number of locations on those components, of first catheter assembly 52. In non-limiting embodiments, first indicator marking 64 can be arranged on first catheter adapter 54, on clamp 77, on luer adapter 79, on first connector 102, and/or on needless access connector 142. Independent of above-described component, first indicator marking can be included at any suitable location on said component. For example, as shown in FIG. 3D, first indicator marking 64 can be arranged on first catheter adapter 54, for example on a wing thereof. In non-limiting embodiments, as shown in FIG. 3G, first indicator marking 64 can be arranged as a marking 64a on piston 145 of needleless access connector 142 and/or a marking 64b on cap 143. In non-limiting embodiments, as shown in FIG. 3J, first indicator marking 64 can be arranged as a marking 64a on an insert associated with clamp 77, as a marking 64b on clamp 77, and/or as a marking 64c on a luer adapter 79.

In some embodiments, a second catheter assembly 66 may include a second catheter adapter 68, which may include a distal end 70, a proximal end 72, and a lumen 74 extending through the distal end 70 and the proximal end 72. In some embodiments, the second catheter assembly 66 may include a second catheter 76 extending from the distal end 70. In some embodiments, the second catheter 76 may include a second length. In some embodiments, the second catheter assembly 66 may include a second indicator marking 78 corresponding to the second length. As shown in the illustrated, non-limiting embodiments of FIGS. 3A-3L, second indicator marking 78 can be arranged on any number of components, and at any number of locations on those components, of second catheter assembly 66. In non-limiting embodiments, second indicator marking 78 can be arranged on second catheter adapter 68, on clamp 77, on luer adapter 79, on second connector 104, and/or on needless access connector 142. Independent of above-described component, first indicator marking can be included at any suitable location on said component. For example, as shown in FIG. 3E, second indicator marking 78 can be arranged on second catheter adapter 68, for example on a wing thereof. In non-limiting embodiments, as shown in FIG. 3H, second indicator marking 78 can be arranged as a marking 78a on piston 145 of needleless access connector 142 and/or a marking 78b on cap 143. In non-limiting embodiments, as shown in FIG. 3K, second indicator marking 78 can be arranged as a marking 78a on an insert associated with clamp 77, as a marking 78b on clamp 77, and/or as a marking 78c on a luer adapter 79.

In some embodiments, a third catheter assembly 80 may include a third catheter adapter 82, which may include a distal end 84, a proximal end 86, and a lumen 88 extending through the distal end 84 and the proximal end 86. In some embodiments, the third catheter assembly 80 may include a third catheter 90 extending from the distal end 84. In some embodiments, the third catheter 90 may include a third length. In some embodiments, the third catheter assembly 80 may include a third indicator marking 92 corresponding to the third length. In some embodiments, the first indicator marking 64, the second indicator marking 78, and/or the third indicator marking 92 may include different indicia and/or different colors, as demonstrated by the shapes and different shading in FIGS. 3A-3L. As shown in the illustrated, non-limiting embodiments of FIGS. 3A-3L, third indicator marking 92 can be arranged on any number of components, and at any number of locations on those components, of third catheter assembly 80. In non-limiting embodiments, third indicator marking 92 can be arranged on third catheter adapter 82, on clamp 77, on luer adapter 79, on third connector 106, and/or on needless access connector 142. Independent of above-described component, first indicator marking can be included at any suitable location on said component. For example, as shown in FIG. 3F, third indicator marking 92 can be arranged on third catheter adapter 82, for example on a wing thereof. In non-limiting embodiments, as shown in FIG. 3I, third indicator marking 92 can be arranged as a marking 92a on piston 145 of needleless access connector 142 and/or a marking 92b on cap 143. In non-limiting embodiments, as shown in FIG. 3L, third indicator marking 92 can be arranged as a marking 92a on an insert associated with clamp 77, as a marking 92b on clamp 77, and/or as a marking 92c on a luer adapter 79.

In some embodiments, a method may include providing one or more of the first catheter assembly 52, the second catheter assembly 66, and the third catheter assembly 80. In some embodiments, the method may include a method of manufacture. In some embodiments, the method may include providing an instrument advancement device 94 configured to couple to one or more of the following: the first catheter assembly 52, the second catheter assembly 66, and the third catheter assembly 80. In some embodiments, the instrument advancement device 94 may be configured to advance an instrument of the instrument advancement device 94 distally into one or more of the first catheter assembly 52, the second catheter assembly 66, and the third catheter assembly 80.

In some embodiments, the instrument advancement device 94 may include a first other indicator marking 96 matching the first indicator marking 64, 64a, and/or 64b and/or a second other indicator marking 98 marking matching the second indicator marking 78, 78a, 78b, and/or 78c. In some embodiments, the instrument advancement device 94 may include a third other indicator marking 100 matching the third indicator marking 92, 92a, and/or 92b.

In some embodiments, one or more of the first indicator marking 64, 64a, and/or 64b, the second indicator marking 78, 78a, 78b, and/or 78c, and the third indicator marking 92, 92a, and/or 92b may include one or more of the following: a color, a color change, a pattern, a symbol, a picture, an icon, an alphanumeric descriptor, an alphabetical descriptor, a numeric descriptor, a line, and another suitable indicator marking. Similarly, in some embodiments, one or more of the first other indicator marking 96, the second other indicator marking 98, and the third other indicator marking 100 may include one or more of the following: a color, a color change, a pattern, a symbol, a picture, an icon, an alphanumeric descriptor, an alphabetical descriptor, a numeric descriptor, a line, and another suitable indicator marking. In some embodiments, the first other indicator marking 96, the second other indicator marking 98, and/or the third other indicator marking 100 may include different colors, as demonstrated by different shading in FIG. 4A.

In some embodiments, the first indicator marking 64, 64a, and/or 64b that matches the first other indicator marking 96 may include one or more of the following in common with the first other indicator marking 96: a color, a color change, a pattern, a symbol, a picture, an icon, an alphanumeric descriptor, an alphabetical descriptor, a numeric descriptor, a line, and another suitable indicator marking. Similarly, in some embodiments, the second indicator marking 78, 78a, 78b, and/or 78c that matches the second other indicator marking 98 may include one or more of the following in common with the second other indicator marking 98: a color, a color change, a pattern, a symbol, a picture, an icon, an alphanumeric descriptor, an alphabetical descriptor, a numeric descriptor, a line, and another suitable indicator marking. Similarly, in some embodiments, the third indicator marking 92, 92a, and/or 92b that matches the third other indicator marking 100 may include one or more of the following in common with the third other indicator marking 100: a color, a color change, a pattern, a symbol, a picture, an icon, an alphanumeric descriptor, an alphabetical descriptor, a numeric descriptor, a line, and another suitable indicator marking. In some embodiments, the alphabetical descriptor may include a word or one or more letters. In some embodiments, a numeric descriptor may include one or more numbers.

In some embodiments, one or more of the first indicator marking 64, 64a, and/or 64b, the second indicator marking 78, 78a, 78b, and/or 78c, the third indicator marking 92, 92a, and/or 98b, the first other indicator marking 96, the second other indicator marking 98, and the third other indicator marking 100 may be stamped, embossed, laser-drilled, two-shot molded, marked, scribed, or created in another suitable manner.

As illustrated in the non-limiting embodiments of FIGS. 3A and 4A, in some embodiments, the first indicator marking 64 may include a same color and/or a same printed length as the first other indicator marking 96. For example, the same printed length may include “1.00” or another suitable number that correspond to a length of the first catheter 62 (in centimeters, millimeters, or another unit). In some embodiments, the second indicator marking 78 may include a same other color (different from the same color of FIG. 3A) and/or a same other printed length (different from the printed length of FIG. 3A) as the second other indicator marking 98. For example, the same other printed length may include “1.25” or another suitable number that correspond to a length of the second catheter 76 (in centimeters, millimeters, or another unit). In some embodiments, the third indicator marking 92 may include a same other color and/or a same other printed length as the third other indicator marking 100. For example, the same other printed length may include “1.75” or another suitable number that correspond to a length of the third catheter 90 (in centimeters, millimeters, or another unit).

In some embodiments, the first catheter assembly 52 may include a first connector 102 coupled to the first catheter adapter 54. In some embodiments, an outer surface of the first connector 102 may include the first indicator marking 64. In some embodiments, the first indicator marking 64 may be disposed on another outer surface of the first catheter assembly 52 that is visible to the clinician. In some embodiments, the second catheter assembly 66 may include a second connector 104 coupled to the second catheter adapter 68. In some embodiments, an outer surface of the second connector 104 may include the second indicator marking 78. In some embodiments, the second indicator marking 78 may be disposed on another outer surface of the second catheter assembly 66 that is visible to the clinician. In some embodiments, the third catheter assembly 80 may include a third connector 106 coupled to the third catheter adapter 82. In some embodiments, an outer surface of the third connector 106 may include the third indicatormarking. In some embodiments, the third indicator marking 92 may be disposed on another outer surface of the third catheter assembly 80 that is visible to the clinician.

In some embodiments, the instrument advancement device 94 may include an advancement feature. In some embodiments, in response to aligning the advancement feature with the first other indicator marking 96, a distal tip of the instrument may be configured to align with the distal tip 44 of the first catheter 62. In some embodiments, in response to aligning the advancement feature with the second other indicator marking 98, the distal tip of the instrument may be configured to align with the distal tip of the second catheter 76. In some embodiments, in response to aligning the advancement feature with the third other indicator marking 100, the distal tip of the instrument may be configured to align with the distal tip of the third catheter 90.

In some embodiments, the advancement feature may include a housing 108. In some embodiments, the instrument advancement device 94 may include the housing 108, a tube 110 extending through the housing 108, a wedge disposed within the housing 108, and a pair of opposing pinch members configured to pinch the tube 110, as described in further detail with respect to FIG. 4E.

Referring now to FIG. 4B, in some embodiments, the first other indicator marking 96 may include a first line having a proximal end 112 and a distal end 114. In some embodiments, the second other indicator marking 98 may include a second line having a proximal end 116 and a distal end 118. In some embodiments, the third other indicator marking 100 may include a third line having a proximal end 120 and a distal end 122.

In some embodiments, in response to aligning the housing 108 (such as a distal end of the housing 108, a line or marking on the housing 108, or another portion of the housing 108) with the proximal end 112 of the first line, the distal tip of the instrument may be configured to align with the distal tip of the first catheter 62. In some embodiments, in response to aligning the housing 108 (such as a distal end of the housing 108, a line or marking on the housing 108, or another portion of the housing 108) with the proximal end 116 of the second line, the distal tip of the instrument may be configured to align with the distal tip of the second catheter 76. In some embodiments, in response to aligning the housing 108 (such as a distal end of the housing 108, a line or marking on the housing 108, or another portion of the housing 108) with the proximal end 120 of the third line, the distal tip of the instrument may be configured to align with the distal tip of the third catheter 90.

In some embodiments, in response to aligning the housing 108 (such as a distal end of the housing 108, a line or marking on the housing 108, or another portion of the housing 108) with the distal end 114 of the first line, the distal tip of the instrument may be configured to extend distal to the distal tip of the first catheter 62. In some embodiments, in response to aligning the housing 108 (such as a distal end of the housing 108, a line or marking on the housing 108, or another portion of the housing 108) with the distal end 118 of the second line, the distal tip of the instrument may be configured to extend beyond the distal tip of the second catheter 76. In some embodiments, in response to aligning the housing 108 (such as a distal end of the housing 108, a line or marking on the housing 108, or another portion of the housing 108) with the distal end 122 of the third line, the distal tip of the instrument may be configured to extend beyond the distal tip of the third catheter 90. Thus, advancement of the instrument may be predictable to the clinician.

Referring now to FIG. 4C, in some embodiments, the tube 110 may include a first lumen 124 and a second lumen 126, which may be separate from the first lumen 124 along an entire length of the tube 110. In some embodiments, a blood collection pathway may extend through the first lumen 124. In some embodiments, the instrument 128 may be disposed within the second lumen 126. In some embodiments, a diameter of the second lumen 126 may be larger than a diameter of the first lumen 124. In some embodiments, the diameter and/or a length of the first lumen 124 may be selected based on a desired flow rate and/or to reduce hemolysis.

In some embodiments, in response to moving the housing 108 distally along the tube 110, the instrument 128 may be advanced distally within the second lumen 126. In some embodiments, in response to moving the housing 108 proximally along the tube 110, the instrument 128 may be retracted proximally within the second lumen 126.

In some embodiments, the instrument advancement device 94 may include a septum 130 disposed within a distal connector 132 and configured to seal the second lumen 126 or prevent blood flow into the second lumen 126. In these and other embodiments, the septum 130 may not seal the first lumen 124 such that blood may flow proximally along a fluid pathway 136 from the distal connector 132 through the first lumen 124 for blood collection. In some embodiments, the septum 130 may be elastomeric. In some embodiments, a fluid pathway for blood collection may be disposed outside of the tube 110. For example, it may extend from the distal connector 132 in an extension tube.

In some embodiments, a distal end of the instrument 128 may be disposed proximal to a distal end of the distal connector 132 when the housing 108 is fully retracted in a proximal direction. In some embodiments, the distal end of the instrument 128 may be disposed proximal to the septum 130 when the housing 108 is fully retracted in the proximal direction and/or the instrument 128 may be sealed within the tube 110.

In some embodiments, the instrument advancement device 94 may include a cannula 134, which may connect a distal end of the first lumen 124 and the distal connector 132. In some embodiments, the cannula 134 may be blunt. In some embodiments, the fluid pathway 136 may extend through the cannula 134, which may prevent blood leakage. In some embodiments, the cannula 134 may be constructed of steel, plastic, metal, or another suitable material. In some embodiments, the cannula 134 may be coupled to the distal connector 132 or monolithically formed with the distal connector 132 as a single unit. In some embodiments, the septum 130 may be concentric with the second lumen 126 or offset slightly to obtain adequate wall thicknesses.

In some embodiments, the distal connector 132 may include a shaft 138 and two lever arms 140. In some embodiments, the two lever arms 140 may facilitate coupling to a needleless access connector 142 (see, for example, FIGS. 3A-3L) or another portion of a particular catheter assembly. In some embodiments, the shaft 138 may be lubricated with a lubricant, which may reduce a force of insertion into the catheter assembly. In some embodiments, the male distal connector may include a male luer, a male luer lock, a male slip luer, a luer, or another suitable connector.

Referring now to FIG. 4D, in some embodiments, the septum 130 may extend across a width of an inner lumen of the distal connector 132. In some embodiments, the septum 130 may seal the second lumen 126 or prevent blood flow into the second lumen 126. In some embodiments, the cannula 134 may extend from the first lumen 124 through the septum 130 to allow fluid flow therethrough.

Referring now to FIG. 4E, in some embodiments, the instrument advancement device 94 may include a wedge 144 disposed within the housing 108 and the second lumen 126 of the tube 110. In some embodiments, the instrument advancement device 94 may include a pair of opposing pinch members 146 configured to pinch the tube 110. In some embodiments, the pair of opposing pinch members 146a,b may be disposed within the housing 108 proximal to the wedge 144 and configured to move along the tube 110 with the housing 108.

In some embodiments, the instrument 128 may be disposed within the second lumen 126. In some embodiments, in response to moving the housing 108 distally along the tube 110, the pair of opposing pinch members 146a,b may push the wedge 144 distally, and the instrument 128 may be advanced distally.

In some embodiments, the instrument advancement device 94 may include anotherpair of opposing pinch members 146c,d configured to pinch the tube 110. In some embodiments, the other pair of opposing pinch members 146c,d may be disposed within the housing distal to the wedge 144 and configured to move along the tube 110 with the housing 108. In some embodiments, in response to moving the housing 108 proximally along the tube 110, the pair of opposing pinch members 146c,d may push the wedge 144 proximally and the instrument 128 may be retracted proximally.

The pair of opposing pinch members 146a,b and the other pair of opposing pinch members 146c,d may be referred to collectively in the present disclosure as “opposing pinch members 146.” In some embodiments, in response to movement of the housing 108 along the tube 110, the opposing pinch members 146 may rotate with respect to the housing 108 and the tube 110. In some embodiments, in response to movement of the housing 108 along the tube 110, the opposing pinch members 146 may rotate with respect to the housing 108 and the tube 110, which may rotate the instrument 128. In some embodiments, an inner surface of the housing 108 may include one or more bumps 148 in contact with the opposing pinch members 146, which may reduce friction as the opposing pinch members 146 rotate. In some embodiments, the wedge 144 and/or the opposing pinch members 146 may be lubricated with a lubricant, which may reduce friction.

In some embodiments, the opposing pinch members 146 may be constructed of plastic, metal, or another suitable material. In some embodiments, the opposing pinch members 146 may include spherical balls, ball bearings, wheels, or cylinders, which may be configured to rotate with respect to the housing 108. In some embodiments, the opposing pinch members 146 may include the wheels, which may be smooth or include feet along their edges. In these embodiments, lubricant may be applied to axels of the wheels to reduce friction. In some embodiments, the opposing pinch members 146 may be fixed with respect to the housing 108. For example, the opposing pinch members 146 may be molded into the housing 108.

In some embodiments, a number of the opposing pinch members 146 may vary based on a shape of the wedge 144. In some embodiments, the instrument advancement device 94 may include the pair of opposing pinch members 146a,b and the other pair of opposing pinch members 146c,d in response to the shape of the wedge 144 being cylindrical, for example. In some embodiments, the instrument advancement device 94 may include a single pair of the opposing pinch members 146, such as the pair of the opposing pinch members 146a,b, in response to the wedge 144 including a dog bone shape, and the single pair may be disposed in a middle or depression of the dog bone shape.

In some embodiments, in response to moving the housing 108 distally along the tube 110, the pair of opposing pinch members 146a,b may push the wedge 144 distally and the instrument 128 may be configured to advanced distally into the first catheter assembly 52, the second catheter assembly 66, and/or the third catheter assembly 80 (see FIGS. 3A-3L). In some embodiments, the first other indicator marking 96, the second other indicator marking 98, and/or the third other indicator marking 100 may be disposed on an outer surface of the tube 110, which may facilitate visibility to the clinician.

In some embodiments, a particular catheter assembly on which the first indicator marking 64, the second indicator marking 78, and/or the third indicator marking 92 are disposed may vary. Also, a particular instrument advancement device on which the first other indicator marking 96, the second other indicator marking 98, and/or the third other indicator marking 100 are disposed may vary.

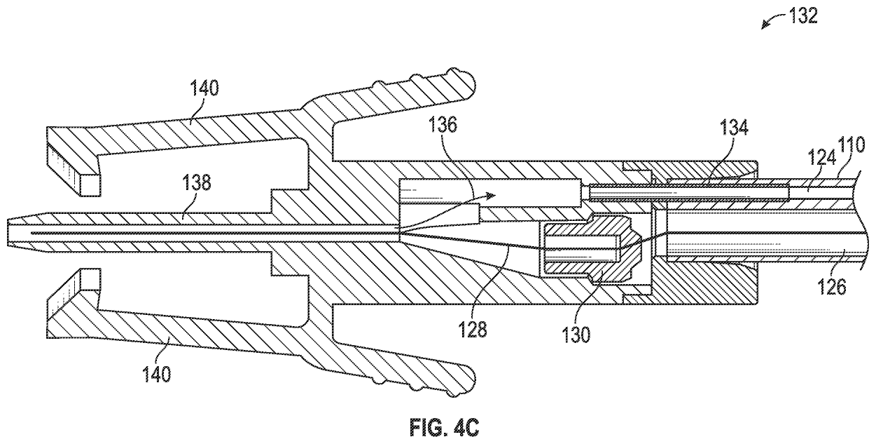

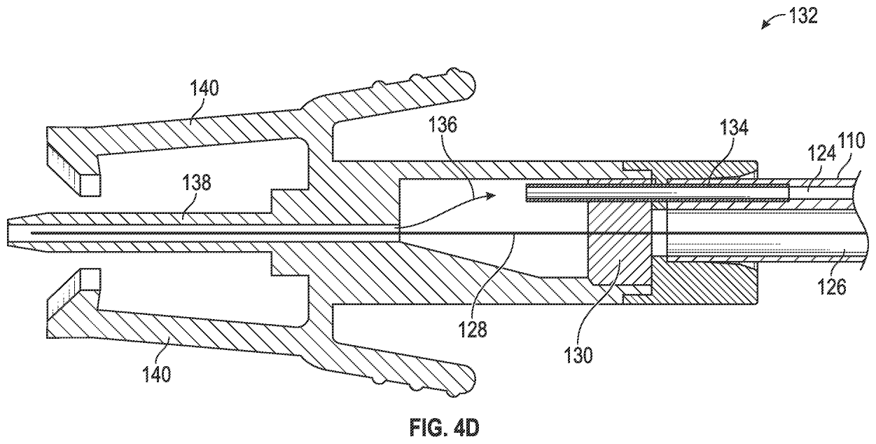

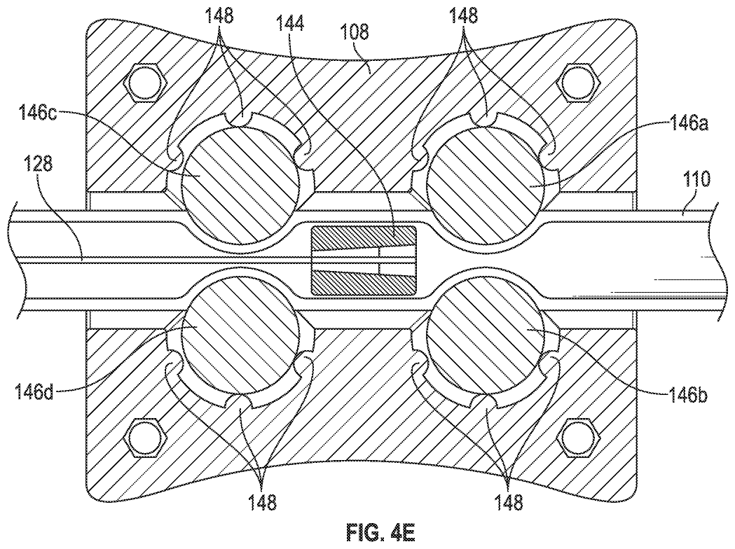

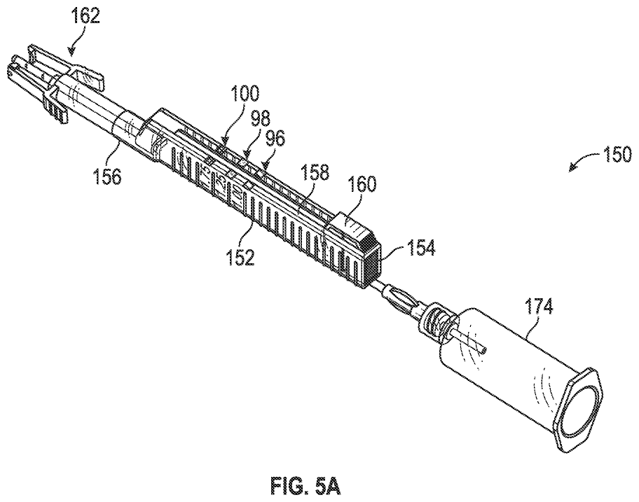

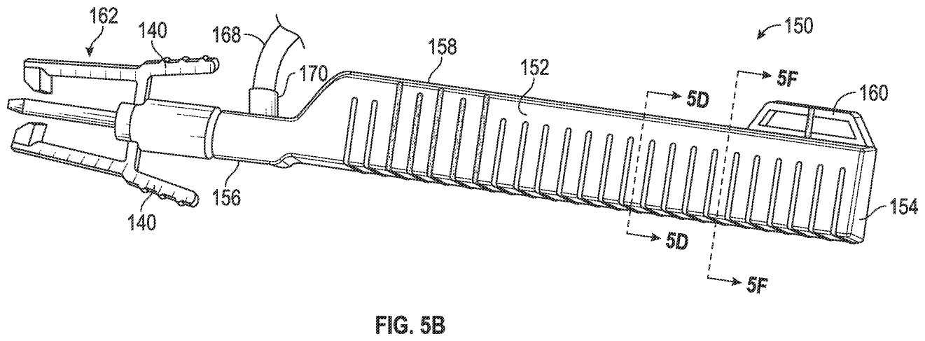

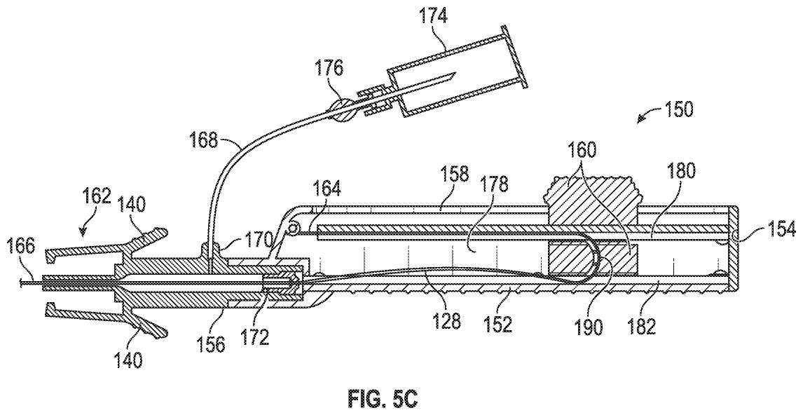

Referring now to FIGS. 5A-5F, an instrument advancement device 150 is illustrated, according to some embodiments. In some embodiments, an instrument advancement device 150 may be configured to deliver the instrument 128 through a catheter of a catheter assembly, such as, for example, the first catheter assembly 52, the second catheter assembly 66, and/or the third catheter assembly 80. In some embodiments, the instrument 128 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 128 may reduce or remove occlusions, improving patency of the catheter for medication and fluid delivery, as well as blood acquisition, during a dwell time of the catheter.

In some embodiments, the instrument advancement device 150 may be similar or identical to the instrument advancement device 94 of FIGS. 4A-4E. In some embodiments, the instrument 128 may include a guidewire, an instrument, a guidewire or an instrument with one or more sensors, or another suitable instrument. In some embodiments, the sensors may be used for patient or device monitoring and may include sensors measuring pressure, temperature, pH, blood chemistry, oxygen saturation, flow rate, or another physiological property. In some embodiments, the catheter through which the instrument 128 may be delivered may have been previously inserted into vasculature of a patient and may be dwelling within the vasculature when the instrument 128 is advanced through the catheter.

In some embodiments, the instrument 128 may be disposed within a housing 152, which may be configured to protect the instrument 128 from damage and/or contamination from a surrounding external environment. In some embodiments, the housing 152 may be rigid or semi-rigid. In some embodiments, the housing 152 may be made of one or more of stainless steel, aluminum, polycarbonate, metal, ceramic, plastic, and another suitable material. In some embodiments, the housing 152 may include a proximal end 154, a distal end 156, and a slot 158. In some embodiments, the slot 158 may extend parallel to a longitudinal axis of the housing 152.

In some embodiments, the instrument advancement device 150 may include an advancement feature 160, which may extend through the slot 158 and may be configured to move linearly along the slot 158 between a retracted position illustrated, for example, in FIGS. 5A-5B, and an advanced position. In some embodiments, the clinician may pinch or grasp the advancement feature 160 to move the advancement feature 160 between the retracted position and the advanced position.

In some embodiments, the distal end 156 of the housing 152 may include a distal connector 162, which may be similar or identical to the distal connector 132 of FIGS. 4C-4D in terms of one or more features and/or operation. In some embodiments, in response to removal of the pressure applied to the proximal ends of the two lever arms, distal ends of the two lever arms may move closer to each other and clasp a portion of the IV catheter device, such as a needleless connector, another connector, or a proximal end of a catheter adapter, for example. In some embodiments, the distal connector 162 may include a blunt cannula or male luer configured to insert into the portion of the IV catheter device.

In some embodiments, the instrument 128 may include a first end 164 and a second end 166, which may include a distal tip. In some embodiments, movement of the advancement feature 160 from the retracted position to the advanced position may cause the second end 166 of the instrument 128 to be advanced beyond the distal end 156 of the housing 152. In some embodiments, moving the advancement feature 160 to the advanced position may introduce the instrument 128 into the IV catheter device.

In some embodiments, an extension tube 168 may be coupled to the instrument advancement device 150, and the extension tube 168 may be used for blood collection and/or fluid infusion. In some embodiments, the extension tube 168 may extend from a port 170 of the housing 152. In some embodiments, a septum 172 may be within the housing 152 to enable the instrument 128 to advance and/or retract while maintaining a closed fluid path. In some embodiments, the instrument 128 may be configured to extend through the septum 172. In some embodiments, the septum 172 may be disposed proximal to the port 170 and distal to the advancement feature 160 in the advanced position. In some embodiments, the septum 172 may include silicone, rubber, an elastomer, or another suitable material. In some embodiments, the septum 172 may include an aperture, slit, or the like to accommodate the instrument 128 therethrough.

In some embodiments, a proximal end of the extension tube 168 may be coupled to a blood collection device 174. For example, the proximal end of the extension tube 168 may be integrated with a connector 176, which may be coupled to the blood collection device 174. In some embodiments, a needleless connector may be disposed between the connector 176 and the blood collection device 174. In some embodiments, the connector 176 and/or the port 170 may be coupled to an IV line or another fluidic connection.

In some embodiments, an inner surface 178 of the housing 152 may include one or more grooves. For example, the inner surface 178 may include a first groove 180 and/or a second groove 182. In some embodiments, the first groove 180 and/or the second groove 182 may be disposed within the housing 152 between the proximal end 154 and the distal end 156. In some embodiments, the instrument 128 may be disposed within the first groove 180 and/or the second groove 182. In some embodiments, the first groove 180 and/or the second groove 182 may include a support wall 184, another support wall 186 opposite the support wall 184, and a bottom 188 extending between the support wall 184 and the other support wall 186. In some embodiments, the first groove 180 and/or the second groove 182 may be open opposite the bottom 188. In some embodiments, the first groove 180 and/or the second groove 182 may be linear and/or configured to guide the instrument 128 as the instrument 128 is advanced distally and/or retracted proximally.

In some embodiments, the instrument 128 may be linear within the housing 152 and the first end 164 of the instrument 128 may be coupled to the advancement feature 160. In other embodiments, the advancement feature 160 may include an arc-shaped channel 190, which may be U-shaped. In some embodiments, the instrument 128 may extend and move through the arc-shaped channel 190. In some embodiments, the first end 164 of the instrument 128 may be fixed. In some embodiments, the first end 164 of the instrument may be fixed within the housing 152. In some embodiments, in response to movement of the advancement feature 160 a first distance, the second end of the instrument 128 may be configured to advance distally a second distance. In some embodiments, the second distance may be twice the first distance. In some embodiments, the second distance may be more than twice the first distance. In these and other embodiments, the instrument 128 may extend through multiple U-shapes or other arc-shapes. In some embodiments, because the first groove 180 and/or the second groove 182 are open opposite the bottom 188, the instrument 128 may tend to buckle in response to the advancement feature 160 being advanced distally, as illustrated, for example, in FIG. 5C. In some embodiments, the first other indicator marking 96, the second other indicator marking 98, and/or the third other indicator marking 100 may be disposed on the housing 152.

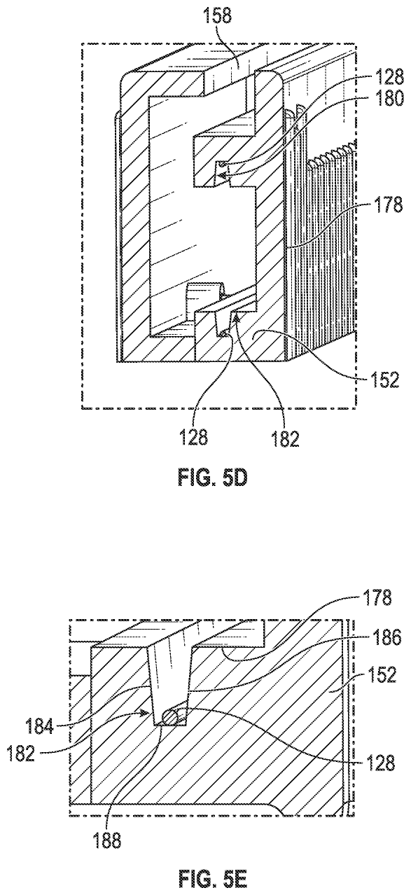

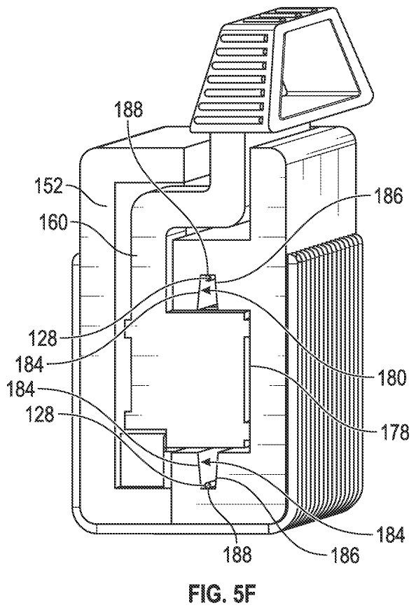

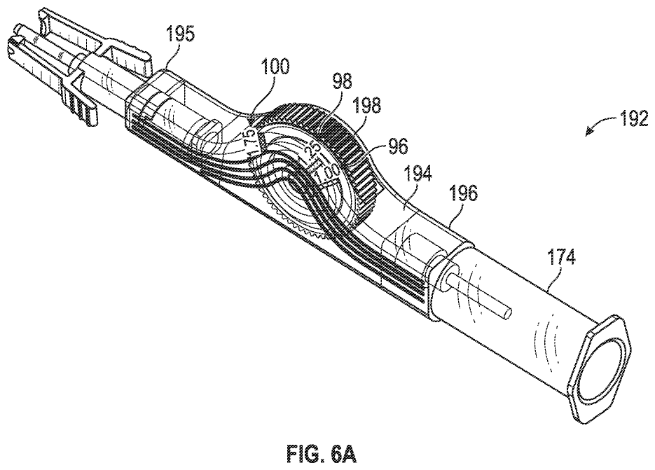

Referring now to FIG. 6A, in some embodiments, an instrument advancement device 192 may include a housing 194, which may include a distal end 195 and a proximal end 196. In some embodiments, the distal end 195 of the housing 194 may be configured to couple to an intravenous catheter device, such as, for example, the first catheter assembly 52, the second catheter assembly 66, and/or the third catheter assembly 80. In some embodiments, the instrument 128 may be disposed within the housing 194.

In some embodiments, the instrument advancement device 192 may be similar or identical to the instrument advancement device 150 of FIGS. 5A-5F and/or the instrument advancement device 94 of FIGS. 4A-4E in terms of one or more features and/or operation. In some embodiments, the instrument advancement device 192 may include an advancement wheel 198, which may correspond to the advancement feature. In some embodiments, the advancement wheel 198 may extend out from the housing 194. In some embodiments, in response to the advancement wheel 198 being rotated, the instrument 128 may be advanced through the distal end 195 of the housing 194. In some embodiments, the first other indicator marking 96, the second other indicator marking 98, and/or the third other indicator marking 100 may be disposed on the housing 194. In some embodiments, the advancement wheel 198 may be rotated such that a marking, such as a line or other marking, on the advancement wheel 198 is aligned with the first other indicator marking 96, the second other indicator marking 98, or the third other indicator marking 100.

Referring now to FIGS. 6B-6C, in some embodiments, the instrument advancement device 192 may include a housing 194 having a distal end 195 and a proximal end 196. In some embodiments, the instrument advancement device 192 may include or correspond to any of the probe delivery devices described in further detail in U.S. Patent Application No. 63/135,393, filed Jan. 8, 2021, and entitled “PROBE DELIVERY DEVICE TO FACILITATE ADVANCEMENT OF A PROBE WITHIN AN INTRAVENOUS CATHETER,” wherein is herein incorporated by reference in its entirety. In some embodiments, although only a portion of the distal end 195 is illustrated, as noted above, the distal end 195 could include any type of connector to enable the instrument advancement device 192 to be connected to an IV catheter device (such as, for example, the first catheter assembly 52, the second catheter assembly 66, and/or the third catheter assembly 80) or could incorporate an IV catheter. In some embodiments, the proximal end 196 may be configured to form a vacuum tube receiver 200 having a cannula 202 covered by a protective sheath 204.

In some embodiments, a fluid pathway 206 may extend within the instrument advancement device 192 from the cannula 202 to the distal end 195. Accordingly, when a vacuum tube is inserted into the vacuum tube receiver 200, a blood sample can be collected through the fluid pathway 206. In some embodiments, the proximal end 196 may include a Luer connector or any other type of connector that is coupled to the fluid pathway 206.

In some embodiments, the instrument advancement device 192 may include an instrument delivery mechanism 208 that enables an instrument 128 to be advanced in a distal direction through an IV catheter and subsequently withdrawn in a proximal direction. In some embodiments, the instrument 128 may include a wire constructed of nickel titanium or another suitable material. In some embodiments, a compartment 210 may be formed within the instrument advancement device 192 and may house the instrument delivery mechanism 208. In some embodiments, a dividing wall 212 may create an instrument channel 214 that extends distally from the compartment 210 and joins the fluid pathway 206 at a distal portion 206a of the fluid pathway 206.

In some embodiments, to isolate the compartment 210 from the fluid pathway 206, a seal 216 (e.g., an elastomeric septum) may be positioned within and span the instrument channel 214. In some embodiments, the instrument 128 may extend through a slit or other opening formed within seal 216. In some embodiments, the seal 216 may provide support to instrument 128 to prevent it from buckling as it is advanced. Although the instrument channel 214 is illustrated as being substantially wider than instrument 128, in some embodiments, dimensions of at least a portion of the instrument channel 214 may be only slightly greater than the instrument 128 so that instrument channel 214 may provide support to prevent buckling of the instrument 128.

In some embodiments, the instrument delivery mechanism 208 may include a spool 218 and the advancement wheel 198, both of which may be configured to rotate within the compartment 210. In some embodiments, the spool 218 may be positioned adjacent to the advancement wheel 198 (i.e., towards the instrument channel 214 relative to the advancement wheel 198). In some embodiments, the advancement wheel 198 may be positioned to extend partially out from the compartment 210 to thereby enable a clinician to use his or her thumb or finger to rotate the advancement wheel 198. In some embodiments, the spool 218 may include a gear 220 having teeth 220a. Likewise, in some embodiments, the advancement wheel 198 may include teeth 222a and may therefore function as a gear. In some embodiments, the teeth 222a may interface with the teeth 220a so that the spool 218 is rotated when the advancement wheel 198 is rotated. In some embodiments, the teeth 222a are formed along the outermost edge of the advancement wheel 198. In other embodiments, however, teeth 222a may be formed along a portion of advancement wheel that is inset relative to the outermost edge.

FIG. 6C provides an exploded rear view of the instrument delivery mechanism 208 in isolation, according to some embodiments. In some embodiments, the spool 218 and the advancement wheel 198 may include axles 224b and 226b, respectively, by which these components are positioned within the compartment 210 and around which these components rotate. In some embodiments, the spool 218 may include a spool drum around which the instrument 128 may be wound. Therefore, when the spool 218 is rotated, the rotation may cause the instrument 128 to be advanced or retracted along the instrument channel 214 depending on the direction in which the advancement wheel 198 is rotated. In some embodiments, the gear formed by the advancement wheel 198 may have a larger diameter than the gear 220 to thereby cause the instrument 128 to be advanced or retracted a larger distance relative to the amount of rotation of the advancement wheel 198. In contrast, in other embodiments, the gear formed by the advancement wheel 198 may have an equal or smaller diameter than the gear 220. In such embodiments, the instrument 128 may advance or retract a smaller distance relative to the amount of rotation of the advancement wheel 198 but such advancement or retraction may be accomplished with a reduced amount of force to the advancement wheel 198.

In some embodiments, the instrument advancement device 192 may include a seal (not illustrated) within the compartment 210 that isolates the spool drum and the instrument 128 from the external environment. In some embodiments, the seal 216 may or may not be employed since fluid entering the instrument channel 214 would be prevented from escaping the compartment 210 by the seal within the compartment 210.

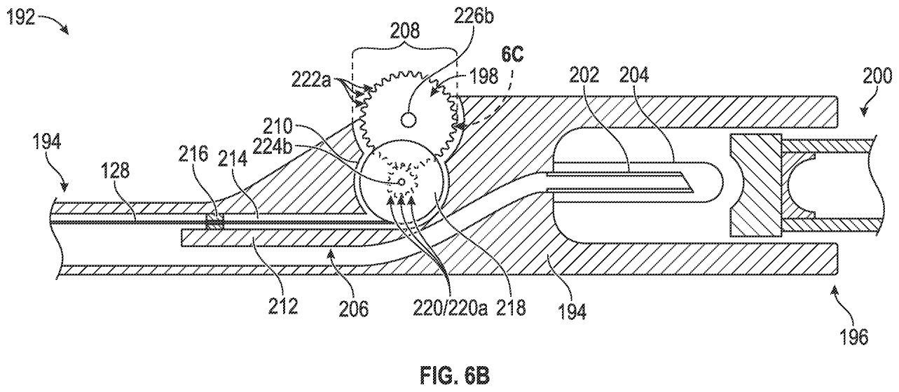

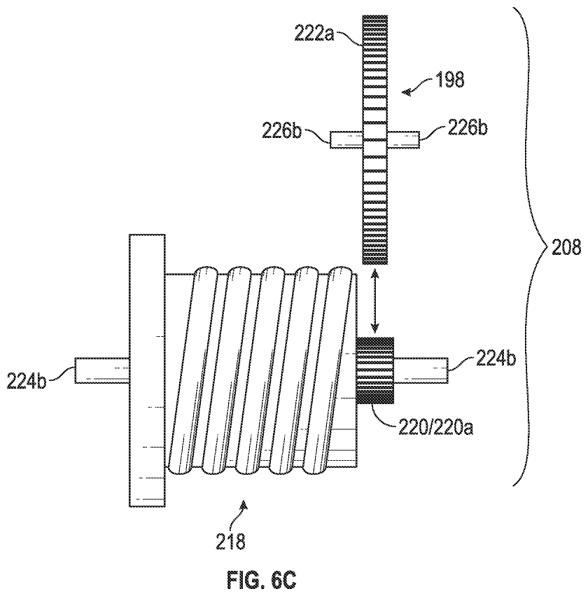

Referring now to FIGS. 7-8, in some embodiments, the second other indicator marking 98 and the third other indicator marking 100 may include a same color, such as, for example, green, and the first other indicator marking 96 may include a different color than the same color, such as, for example, red. In some embodiments, a color change may indicate passing of the distal tip of a particular catheter by the instrument 128.

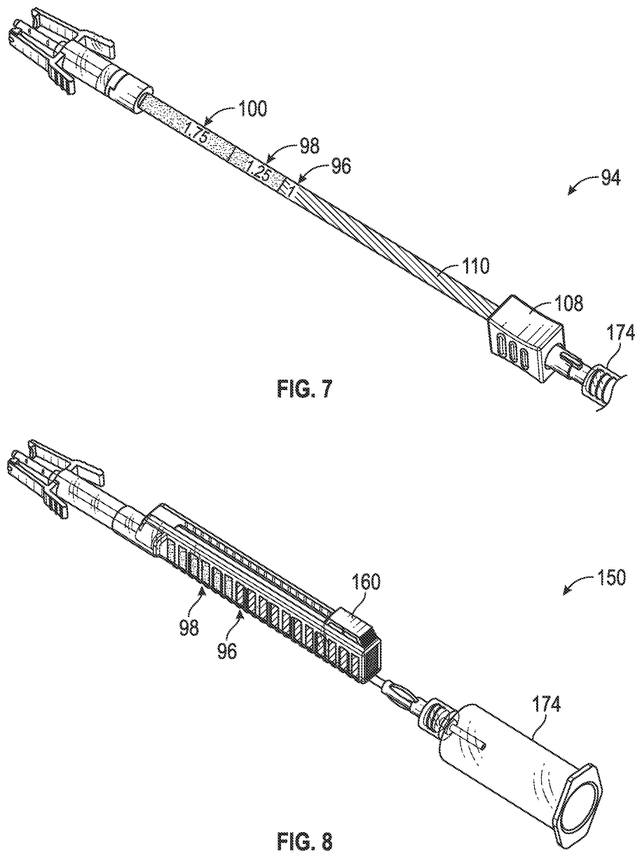

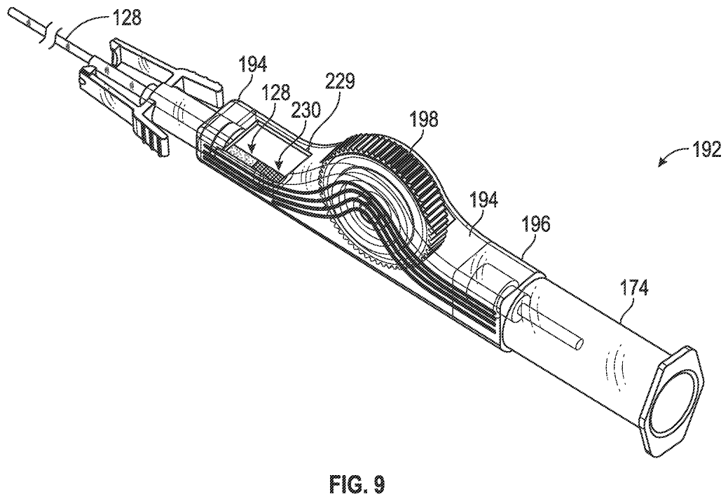

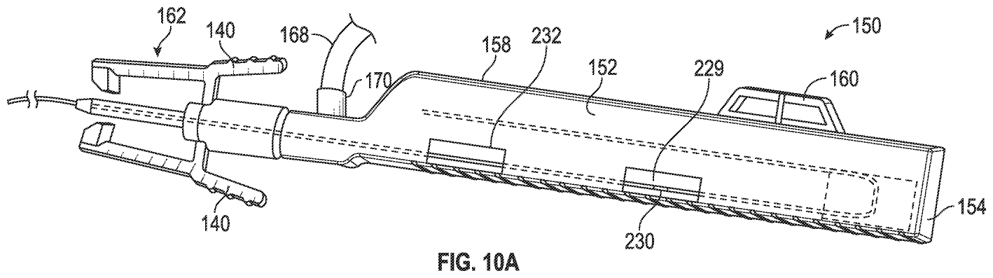

Referring now to FIGS. 9 and 10A, in some embodiments, a particular instrument advancement device may include one or more windows that may allow the clinician to observe an indicator marking 230 on the instrument 128 to determine a position of the distal tip of a catheter. In some embodiments, the indicator marking 230 may be similar or identical to the first other indicator marking 96, for example. In some embodiments, the indicator marking 230 may include one or more of the following: a color, a color change, a pattern, a symbol, a picture, an icon, an alphanumeric descriptor, an alphabetical descriptor, a numeric descriptor, a line, and another suitable indicator marking.

As illustrated in FIG. 9, in some embodiments, the instrument advancement device 192 may include a window 229, which may include glass or another suitable transparent material. In some embodiments, in response to the advancement wheel 198 being rotated, the indicator marking 230 may be disposed within the window 229, and the distal tip of the instrument 128 may be advanced distally through the distal end of the housing 194 a distance.

As illustrated in FIG. 10A, in some embodiments, the instrument advancement device 150 may include the window 229 and a window 232, which may each include glass or another suitable transparent material. In some embodiments, the window 232 may be aligned with the window 229 and/or distal to the window 229. In some embodiments, in response to movement of the indicator marking 230 to a position within the window 229, a distal tip of the instrument 128 may be advanced distally a distance. In some embodiments, in response to the indicator marking being within the window 229, the distal tip of the instrument 128 may be advanced distally another distance. In some embodiments, the other distance may be greater than the distance.

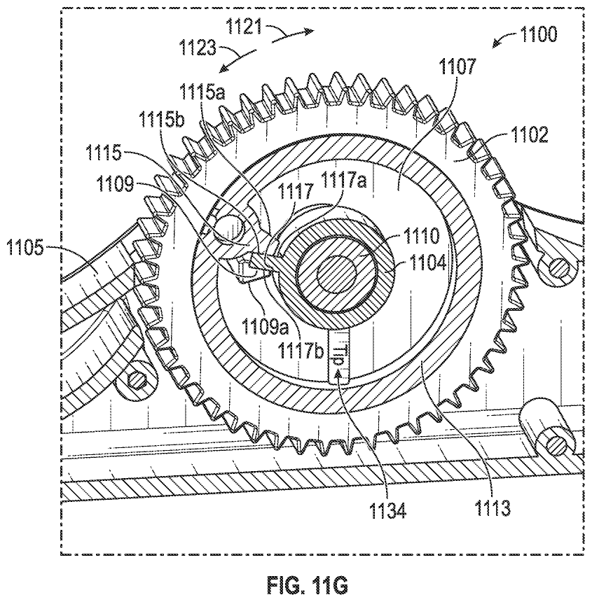

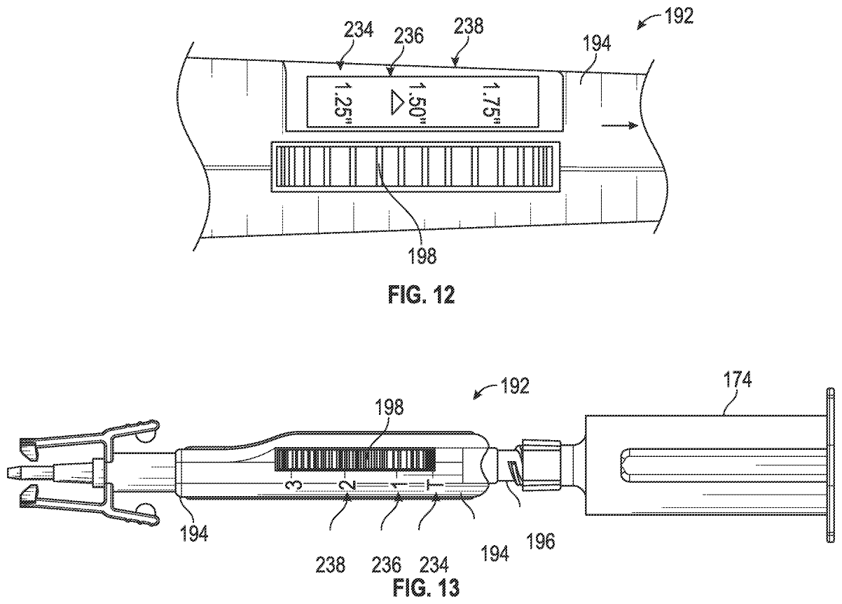

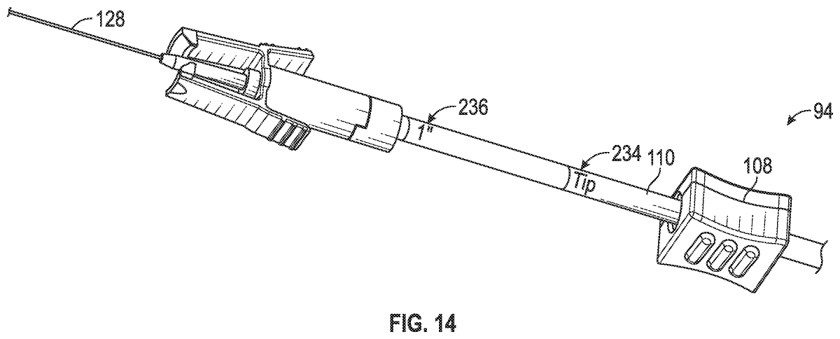

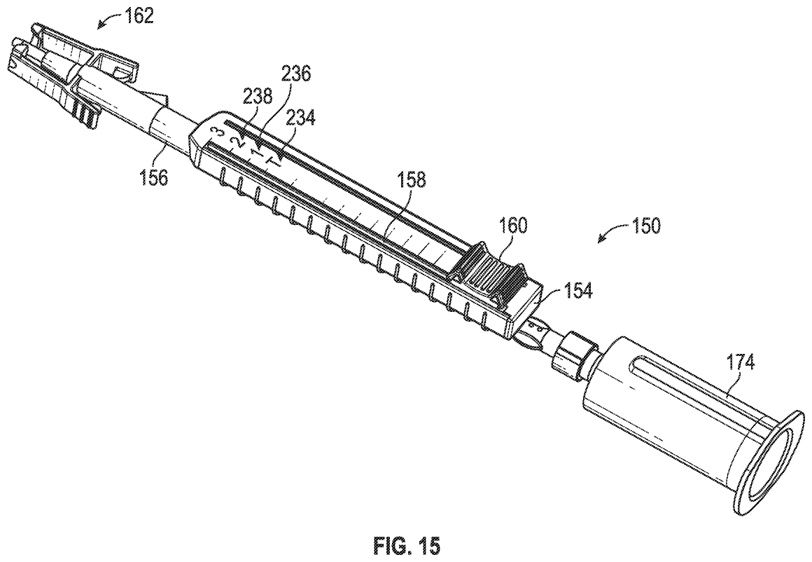

Referring now to FIGS. 10B and 12-15, a particular instrument advancement device may include one or more of the following: a first indicator marking 234, a second indicator marking 236, and a third indicator marking 238. In some embodiments, the particular instrument advancement device may include one or more other indicator markings. In some embodiments, in response to aligning the advancement feature with the first indicator marking 234, the distal tip of the instrument 128 may be configured to align with a distal tip of a particular catheter of a particular catheter assembly to which the particular instrument advancement device may be coupled.

In some embodiments, in response to aligning the advancement feature with the second indicator marking 236, the distal tip of the instrument 128 may be advanced a distance distal to the distal tip of the particular catheter. In some embodiments, in response to aligning the advancement feature with the third indicator marking 238, the distal tip of the instrument 128 may be advanced another distance distal to the distal tip of the particular catheter, which may be greater than the distance. In some embodiments, the first indicator marking 234, the second indicator marking 236, and the third indicator marking 236 may be similar or identical to the first other indicator marking 96, the second other indicator marking 98, and the third other indicator marking 100, respectively, in terms of one or more features and/or operation.

In some embodiments, the first indicator marking 234 may include a printed T or a printed word “Tip”. In some embodiments, the second indicator marking 236 may include a printed number corresponding to the distance. In some embodiments, the third indicator marking 238 may include a printed number corresponding to the other distance. In some embodiments, one or more of the first indicator marking 234, the second indicator marking 236, and the third indicator marking 238 may include one or more of the following: a color, a color change, a pattern, a symbol, a picture, an icon, an alphanumeric descriptor, an alphabetical descriptor, a numeric descriptor, line, and another suitable indicator marking.

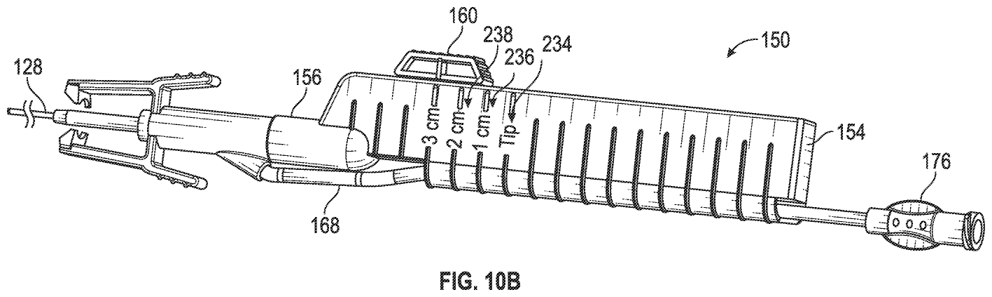

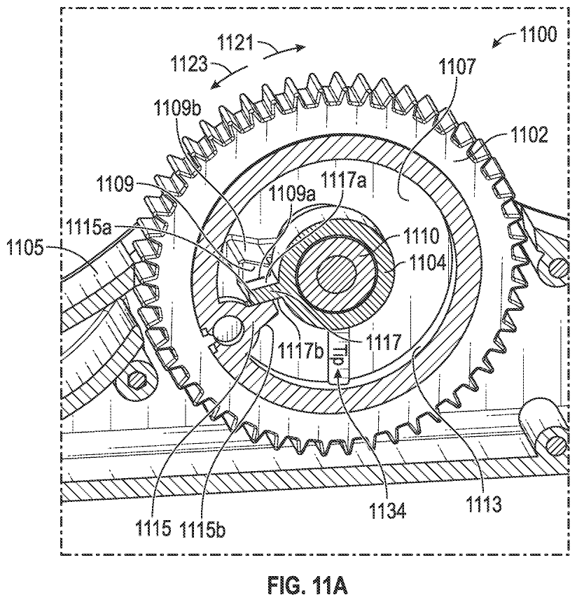

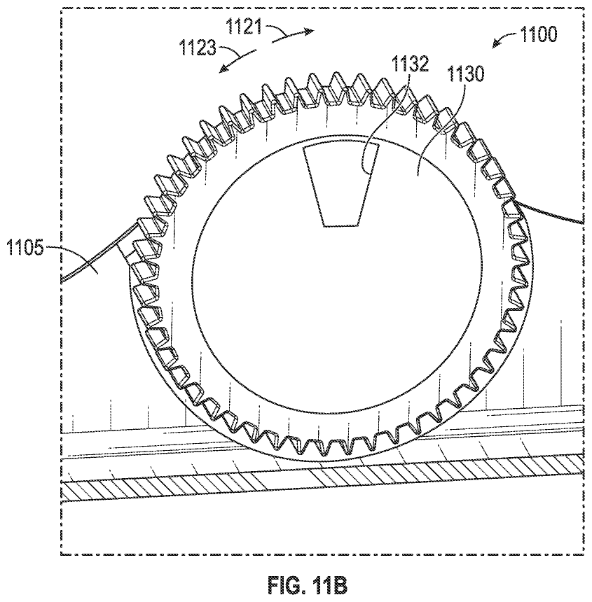

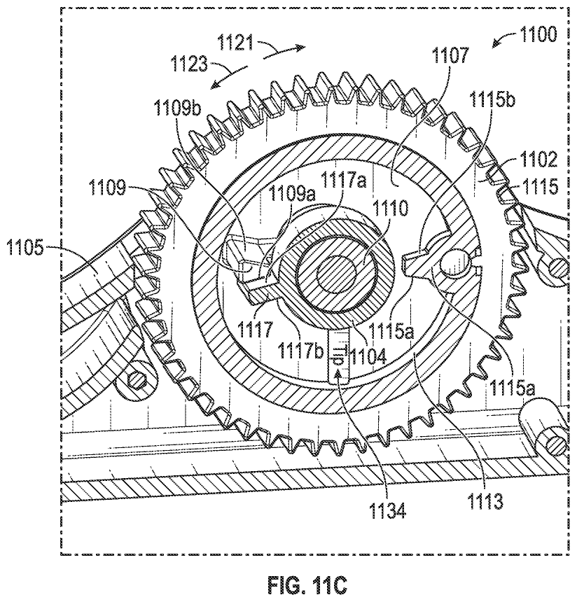

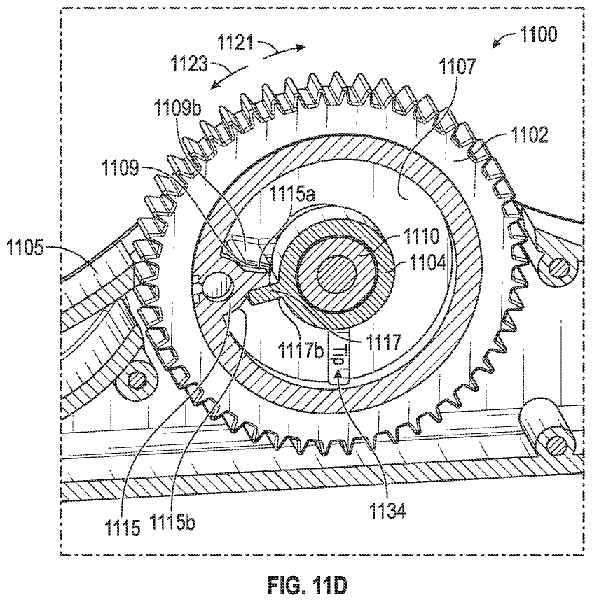

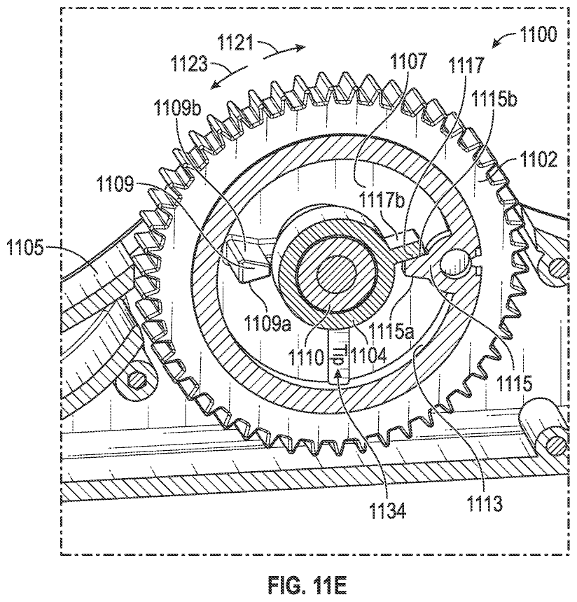

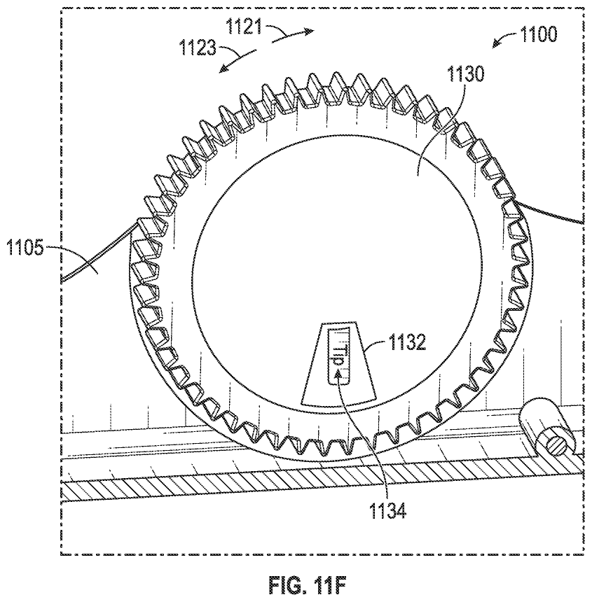

Referring now to FIG. 11, an instrument advancement device 1100 is illustrated, in accordance with some embodiments. In some embodiments, the instrument advancement device 1100 may be similar or identical to the instrument advancement device 192 or another instrument advancement device that includes an advancement wheel. In some embodiments, the instrument advancement device 1100 may include a first wheel 1102 and a second wheel 1104. In some embodiments, an inner surface 1107 of the housing 1105 may include a housing stop member 1109, which may include a protrusion. In some embodiments, the housing stop member 1109 may include a first side 1109a and a second side 1109b, which may be opposite the first side 1109a.

In some embodiments, the first wheel 1102 may rotate on an axle 1111. In some embodiments, the first wheel 1102 may include a protrusion or rod 1110, which may rotate on the axle 1111. In some embodiments, the axle 1111 may be aligned with a center axis of the first wheel 1102. In some embodiments, the second wheel 1104 may be disposed on the rod 1110 and configured to slip with respect to the rod 1110 and/or rotate independently from the rod 1110. In some embodiments, the second wheel 1104 may ride directly on the axle 1111, along with the first wheel 1102. In some embodiments, the axle 1111 may extend inwardly from the housing 1105. In some embodiments, the first wheel 1102 and the second wheel 1104 may rotate about a same axis and/or the second wheel 1104 may be disposed within the first wheel 1102.

In some embodiments, an inner surface 1113 of the first wheel 1102 may include a first wheel stop member 1115, which may include a protrusion. In some embodiments, a gap may be disposed between the housing stop member 1109 and the first wheel stop member 1115. In some embodiments, the second wheel 1104 may include a tab 1117, which may be configured to bridge the gap between the housing stop member 1109 and the first wheel stop member 1115. In some embodiments, the instrument advancement device 1100 may include an instrument, such as, for example, the instrument 128.

In some embodiments, the first wheel 1102 may be configured to rotate to advance the instrument in a distal direction through the distal end of the housing 1105. In some embodiments, the first wheel 1102 may be configured to rotate more than one full turn. In some embodiments, the instrument advancement device 1100 may be disposed in a first configuration, as illustrated, for example, in FIG. 11A.

In some embodiments, in response to the instrument advancement device 1100 being disposed in the first configuration, the tab 1117 may be disposed between the housing stop member 1109 and the first wheel stop member 1115 and may contact the housing stop member 1109 and the first wheel stop member 1115. In these embodiments, a first side 1117a of the tab 1117 may contact a first side 1109a of the housing stop member 1109 and a second side 1117b of the tab 1117 may contact a first side 1115a of the first wheel stop member 1115. In some embodiments, in response to the instrument advancement device 1100 being disposed in the first configuration, the first wheel 1102 and/or the second wheel 1104 may be prevented from rotating in a first direction 1121, toward the housing stop member 1109, but may be configured to rotate in a second direction 1123 opposite the first direction 1121. In some embodiments, the first wheel 1102 may be configured to rotate more than one full turn in the second direction.

In some embodiments, in response to the instrument advancement device 1100 being disposed in the first configuration, the first wheel 1102 may be configured to rotate independent from the housing 1105 and the second wheel 1104 in a second direction 1123 until the first wheel stop member 1115 contacts the tab 1117. In these embodiments, the first wheel 1102 may be configured to rotate independent from the housing 1105 and the second wheel 1104 in the second direction 1123 opposite the first direction 1121 until a second side 1115b of the first wheel stop member 1115 contacts the first side 1117a of the tab 1117, which may be disposed on an opposite side of the tab 1117 as the second side 1117b. In these embodiments, the first wheel 1102 may be configured to rotate almost one full turn independent from the housing 1105 and the second wheel 1104.

In some embodiments, the instrument may be in a retracted or fully retracted position in response to the instrument advancement device 1100 being in the first configuration. In some embodiments, in response to the first wheel 1102 rotating independent from the housing 1105 and the second wheel 1104 in the second direction 1123 until the first wheel stop member 1115 contacts the second side 1117b of the tab 1117, the first wheel 1102 and the second wheel 1104 are configured to rotate together further in the second direction 1123 until the instrument advancement device 1100 is disposed in a second configuration. In these embodiments, the first wheel 1102 and the second wheel 1104 may be configured to rotate together almost one full turn.

In some embodiments, the instrument may be in an advanced or fully advanced position in response to the instrument advancement device 1100 being in the second configuration. In some embodiments, in the second configuration, the tab 1117 may be disposed between the housing stop member 1109 and the first wheel stop member 1115 and may contact the housing stop member 1109 and the first wheel stop member 1115. In these embodiments, the second side 1117b of the tab 1117 may contact the second side 1109b of the housing stop member 1109 and the first side 1117a of the tab 1117 may contact the second side 1115b of the first wheel stop member 1115, as illustrated, for example, in FIG. 11G.

In some embodiments, FIG. 11C illustrates the first wheel 1102 rotating in the second direction 1123 independently from the first configuration. In some embodiments, the first wheel 1102 may rotate in the second direction 1123 independently from the first configuration until the first side 1115a of the first wheel stop member 1115 contacts the first side 1117a of the tab 1117, as illustrated in FIG. 11D, for example. In some embodiments, FIG. 11E illustrates the first wheel 1102 and the second wheel 1104 may rotate together further in the second direction 1123, as illustrated in FIG. 11D, for example. In some embodiments, the first wheel 1102 and the second wheel 1104 may rotate together until the second side 1117b of the tab 1117 contacts the second side 1109b of the housing stop member 1109, as illustrated, for example, in FIG. 11E, preventing further rotation in the second direction 1123.

In some embodiments, the first wheel 1102 may be configured to rotate almost two full turns, from the first configuration to the second configuration. In is understood that in some embodiments, the instrument advancement device 1100 one or more other wheels that operate in a similar fashion to the second wheel 1104 to each allow almost another full turn of the first wheel 1102. In these embodiments, the one or more other wheels may be disposed between the second wheel 1104 and the housing stop member 1109.

In some embodiments, the first wheel 1102 may extend out from the housing 1105, which may facilitate turning of the first wheel 1102 by a digit of the clinician. In some embodiments, in order to advance the instrument, the clinician may rotate the portion of the first wheel 1102 exposed from the housing 1105 toward the distal end 1100a of the housing 1105 or in the second direction 1123 to advance the instrument distally. In some embodiments, the clinician may rotate the portion of the first wheel 1102 exposed from the housing 1105 away from the distal end 1100a of the housing 1105 or in the first direction 1121 to retract the instrument proximally.

It is understood, however, in some embodiments, positions of the housing stop member 1109 and the first wheel stop member 1115 may be reversed. In these embodiments, the clinician may rotate the portion of the first wheel 1102 exposed from the housing 1105 away from the distal end 1100a of the housing 1105 or in the first direction 1121 to advance the instrument distally and/or the clinician may rotate the portion of the first wheel 1102 exposed from the housing 1105 away from the distal end 1100a of the housing 1105 or in the first direction 1121 to retract the instrument proximally. In some embodiments, a location of the housing stop member 1109, the tab 1117, and the first wheel stop member 1115 in the first configuration may vary.

In some embodiments, additional geometry can be added to the first wheel 1102 and/or housing 1105 so there would be a detent at a beginning and/or an end of rotation or travel of the first wheel 1102. In some embodiments, multiple detents may act against the second wheel 1104, and the second wheel 1104 may slide axially to allow one detent to act at a time. The additional geometry may include different ramp angles to encourage one ramp to act before the other.

In some embodiments, the instrument advancement device 1100 may include a cover 1130 coupled to the second wheel 1104 and configured to rotate with the second wheel 1004. In some embodiments, the cover 1130 may include a window 1132, which may be constructed of glass or another suitable transparent material. In some embodiments, when the instrument advancement device 1100 is in the first configuration, the cover 1130 may hide an indicator marking 1134 disposed on an inner surface of the housing 1105, as illustrated, for example, in FIG. 11B. In some embodiments, in response to rotation of the second wheel 1104 in the second direction from the first configuration, the indicator marking 1134 may be disposed in the window 1132, as illustrated, for example, in FIG. 11F. In some embodiments, the indicator marking 1134 may be similar or identical to the first indicator marking 234, the second indicator marking 236, or third indicator marking 238 in terms of one or more features and/or operation.

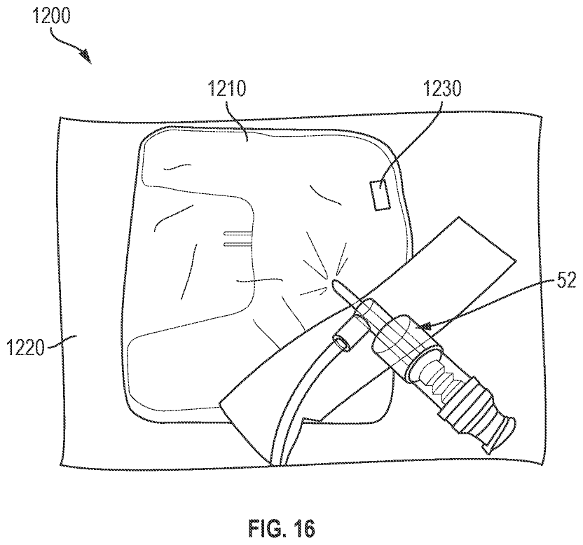

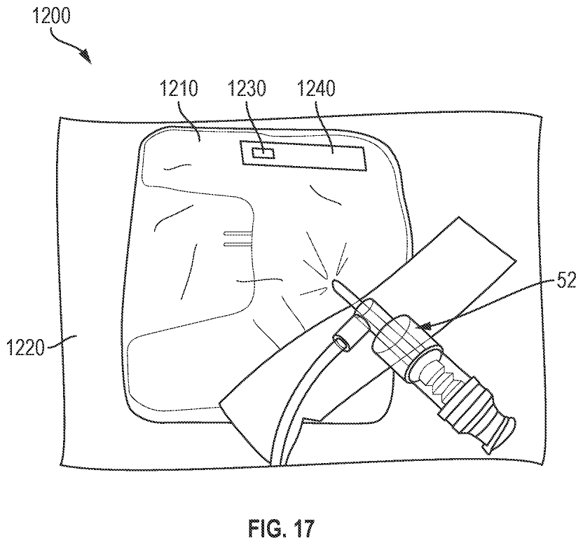

Referring now to FIGS. 16 and 17, non-limiting embodiments of a wound cover 1200 for use with a catheter assembly, such as catheter assemblies 52, 66, 80, is illustrated. Wound cover 1200 can be arranged at a location on a patient's skin where a catheter, such as first catheter 62, second catheter 76, and/or third catheter 90, pierce the patient's skin and enter the vasculature. Wound cover 1200 can include a dressing 1210, formed of any suitable material known to those of skill in the art. In non-limiting embodiments or aspects, wound cover 1200 can include a seal 1220, formed of any suitable material and including, in non-limiting embodiments, an adhesive on at least one side thereof, which can be arranged over dressing 1210 to, for example, isolate the location of the venipuncture from ambient environment.

In non-limiting embodiments, for example as shown in FIG. 16, dressing 1210 and/or seal 1220 can include one or more indicator markings 1230, indicating a length of a catheter as described herein. Arranging the one or more indicator markings 1230 on dressing 1210 and/or cover 1200 can allow a user to visualize a length of the catheter (e.g., 62, 76, and/or 90) in instances where dressing 1210 covers one or more portions of a catheter assembly (e.g., 52, 66, 80) and potentially obscures one or more of indicator markings thereon. As described in greater detail above, one or more indicator markings 1230 can be associated with a matching marking (e.g., 96, 98, 100, 230, 234, 236, 238, and/or 1134) on another medical device, such as instrument advancement device 94, 150, and/or 192.

Turning to FIG. 17, in non-limiting embodiments, a kit including wound cover 1200 (including one or both of dressing 1210 and seal 1220), and, optionally, a catheter assembly (e.g., 52, 66, and/or 80), can include a label 1240 having one or more indicator markings 1230 thereon. Label 1240 can include an adhesive on one side thereof, to allow the label 1240 to be applied to wound cover 1200 and allow a user to visualize a length of the catheter (e.g., 62, 76, and/or 90) in instances where dressing 1210 covers one or more portions of a catheter assembly (e.g., 52, 66, 80) and potentially obscures one or more of indicator markings thereon.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A method of manufacture, comprising:
providing a first catheter assembly, wherein the first catheter assembly comprises:
a first catheter adapter, comprising a distal end, a proximal end, and a lumen extending through the distal end and the proximal end;
a first catheter extending from the distal end of the first catheter adapter, wherein the first catheter comprises a first length; and
a first indicator marking corresponding to the first length;
providing a second catheter assembly, wherein the second catheter assembly comprises:
a second catheter adapter, comprising a distal end, a proximal end, and a lumen extending through the distal end and the proximal end;
a second catheter extending from the distal end of the second catheter adapter, wherein the second catheter comprises a second length; and
a second indicator marking corresponding to the second length;
providing an instrument advancement device configured to couple to the first catheter assembly and the second catheter assembly, wherein the instrument advancement device is configured to advance an instrument distally into the first catheter assembly and the second catheter assembly, wherein the instrument advancement device comprises a first other indicator marking matching the first indicator marking and a second other indicator marking matching the second indicator marking.

2. The method of claim 1, wherein the first catheter assembly further comprises a first connector coupled to the first catheter adapter, wherein an outer surface of the first connector comprises the first indicator marking, wherein the second catheter assembly further comprises a second connector coupled to the second catheter adapter, wherein an outer surface of the second connector comprises the second indicator marking.

3. The method of claim 1, wherein the instrument advancement device comprises an advancement feature, wherein in response to aligning the advancement feature with the first other indicator marking, a distal tip of the instrument is configured to align with a distal tip of the first catheter, wherein in response to aligning the advancement feature with the second other indicator marking, the distal tip of the instrument is configured to align with the distal tip of the second catheter.

4. The method of claim 1, further comprising providing a third catheter assembly, wherein the third catheter assembly comprises:
a third catheter adapter, comprising a distal end, a proximal end, and a lumen extending through the distal end and the proximal end;
a third catheter extending from the distal end of the third catheter adapter, wherein the third catheter comprises a third length; and
a third indicator marking corresponding to the third length, wherein the instrument advancement device comprises a third other indicator marking matching the third indicator marking.

5. The method of claim 4, wherein the instrument advancement device comprises an advancement feature, wherein in response to aligning the advancement feature with the third other indicator marking, a distal tip of the instrument is configured to align with a distal tip of the third catheter.

6. The method of claim 4, wherein the second other indicator marking and the third other indicator marking comprise a same color, wherein the first other indicator marking comprises a different color than the same color.

7. The method of claim 1, wherein the first indicator marking comprises a same color and/or a same printed length as the first other indicator marking, wherein the second indicator marking comprises a same other color and/or a same other printed length as the second other indicator marking.

8. The method of claim 1, wherein the instrument advancement device comprises:
a housing;
an tube extending through the housing; a wedge disposed within the housing;
a pair of opposing pinch members configured to pinch the tube, wherein the pair of opposing pinch members are disposed within the housing and configured to move along the tube with the housing;
the instrument disposed within the tube, wherein in response to moving the housing distally along the tube, the pair of opposing pinch members push the wedge distally and the instrument is configured to advanced distally into the first catheter assembly and the second catheter assembly, wherein the first other indicator marking and the second other indicator marking are disposed on the tube.

9. The method of claim 8, wherein the first other indicator marking comprises a first line having a proximal end and a distal end, wherein the second other indicator marking comprises a second line having a proximal end and a distal end, wherein in response to aligning the housing with the proximal end of the first line, a distal tip of the instrument is configured to align with a distal tip of the first catheter, wherein in response to aligning the housing with the proximal end of the second line, the distal tip of the instrument is configured to align with the distal tip of the second catheter, wherein in response to aligning the housing with the distal end of the first line, the distal tip of the instrument is configured to extend distal to the distal tip of the first catheter, wherein in response to aligning the housing with the distal end of the second line, the distal tip of the instrument is configured to extend beyond the distal tip of the second catheter.

10. The method of claim 1, wherein the instrument advancement device comprises:
a housing, comprising a proximal end, a distal end, and a slot;
an advancement feature extending through the slot and configured to move linearly along the slot between a retracted position and an advanced position; and
the instrument comprising a first end and a second end, wherein in response to movement of the advancement feature from the retracted position to the advanced position, the second end of the instrument is advanced beyond the distal end of the housing, wherein the first other indicator marking and the second other indicator marking are disposed on the housing.

11. The method of claim 1, wherein the instrument advancement device comprises:
a housing, comprising a distal end and a proximal end, wherein the distal end is configured to couple to an intravenous catheter device;
the instrument disposed within the housing; and
an advancement wheel, wherein the advancement wheel extends out from the housing, wherein in response to the advancement wheel being rotated, the instrument is advanced through the distal end of the housing, wherein the first other indicator marking and the second other indicator marking are disposed on the housing.

12. An instrument advancement device, comprising:
an instrument, wherein the instrument comprises an indicator marking; and
a window, wherein in response to movement of the indicator marking to a position within the window, a distal tip of the instrument is advanced distally a distance.

13. The instrument advancement device of claim 12, further comprising another window aligned with and distal to the window, wherein in response to the indicator marking being within the window, the distal tip of the instrument is advanced distally another distance, wherein the other distance is greater than the distance.

14. The instrument advancement device of claim 13, further comprising: a housing;
a tube extending through the housing; a wedge disposed within the housing;
a pair of opposing pinch members configured to pinch the tube, wherein the pair of opposing pinch members are disposed within the housing and configured to move along the tube with the housing, wherein the window is disposed within the tube, wherein the instrument is disposed within the tube, wherein in response to moving the housing distally along the tube, the pair of opposing pinch members push the wedge distally, the indicator marking is disposed within the window, and the distal tip of the instrument is advanced distally a distance beyond the tube, wherein in response to moving the housing distally further along the tube the pair of opposing pinch members push the wedge distally, the indicator marking is disposed within the other window, and the distal tip of the instrument is advanced distally another distance beyond the tube, wherein the other distance is greater than the distance.

15. The instrument advancement device of claim 12, further comprising:
a housing, comprising a distal end and a proximal end, wherein the distal end is configured to couple to an intravenous catheter device, wherein the window is disposed within the housing;
an instrument disposed within the housing; and
an advancement wheel, wherein the advancement wheel extends out from the housing, wherein in response to the advancement wheel being rotated:
the indicator marking is disposed within the window; and
the distal tip of the instrument is advanced distally through the distal end of the housing a distance.

16. An instrument advancement device configured to couple to a catheter assembly, the instrument advancement device comprising:
an instrument;
an advancement feature configured to advance the instrument distally through the catheter assembly;
a first indicator marking;
a second indicator marking;
a third indicator marking, wherein in response to aligning the advancement feature with the first indicator marking, a distal tip of the instrument is configured to align with a distal tip of a catheter assembly, wherein in response to aligning the advancement feature with the second indicator marking, the distal tip of the instrument is advanced a distance distal to the distal tip of the catheter assembly, wherein in response to aligning the advancement feature with the third indicator marking, the distal tip of the instrument is advanced another distance distal to the distal tip of the catheter assembly.

17. The instrument advancement device of claim 16, wherein the first indicator marking comprises a printed T or a printed word “Tip”, wherein the second indicator comprises a printed number corresponding to the distance, wherein the third indicator comprises a printed number corresponding to the other distance.

18. The instrument advancement device of claim 16, wherein the instrument advancement device further comprises:
a housing, comprising a proximal end, a distal end, and a slot, wherein the instrument is disposed within the housing, wherein the advancement feature extends through the slot and is configured to move linearly along the slot between a retracted position and an advanced position, wherein in response to movement of the advancement feature from the retracted position to the advanced position, the second end of the instrument is advanced beyond the distal end of the housing, wherein the first other indicator marking, the second other indicator marking, and the third other indicator marking are disposed on the housing.

19. The instrument advancement device of claim 16, wherein the instrument advancement device further comprises a housing, comprising a distal end and a proximal end, wherein the distal end of the housing is configured to couple to an intravenous catheter device, wherein the instrument is disposed within the housing, wherein the advancement feature comprises an advancement wheel, wherein the advancement wheel extends out from the housing, wherein in response to the advancement wheel being rotated, the instrument is advanced through the distal end of the housing, wherein the first other indicator marking, wherein the second other indicator marking, and the third other indicator marking are disposed on the housing.

20. The instrument advancement device of claim 16, further comprising:
a housing;
a tube extending through the housing; a wedge disposed within the housing;
a pair of opposing pinch members configured to pinch the tube, wherein the pair of opposing pinch members are disposed within the housing and configured to move along the tube with the housing, wherein the instrument is disposed within the tube, wherein in response to moving the housing distally along the tube, the pair of opposing pinch members push the wedge distally and the instrument configured to advanced distally into the first catheter assembly and the second catheter assembly, wherein the first other indicator marking, the second other indicator marking, and the third other indicator marking are disposed on the tube.

21. An instrument advancement device, comprising:
a housing, comprising a distal end and a proximal end, wherein the distal end is configured to couple to an intravenous catheter device, wherein an inner surface of the housing comprises a housing stop member and an indicator marking;
a first wheel, wherein an inner surface of the first wheel comprises a first wheel stop; and a second wheel, wherein the second wheel comprises a tab, wherein in response to the instrument advancement device being disposed in a first configuration, the first wheel and the second wheel are prevented from rotating in a first direction but are configured to rotate in a second direction opposite the first direction, wherein the first wheel is configured to rotate more than one full turn in the second direction;
a cover coupled to the second wheel and configured to rotate with the second wheel, wherein the cover comprises a window, wherein when the instrument advancement device is in the first configuration, the cover hides the indicator marking, wherein in response to rotation of the second wheel in the second direction from the first configuration, the indicator marking is disposed in the window.

22. A method of manufacture, comprising:
providing a first catheter assembly, wherein the first catheter assembly comprises:
a first catheter adapter, comprising a distal end, a proximal end, a side port disposed between the distal end of the first catheter adapter and a proximal end of the first catheter adapter, and a lumen extending through the distal end of the first catheter adapter and the proximal end of the first catheter adapter;
a first catheter extending from the distal end of the first catheter adapter, wherein the first catheter comprises a first catheter length;
a first extension tube extending from the side port of the first catheter adapter, wherein the first extension tube comprises a first extension tube length; and
a first connector coupled to a proximal end of the first extension tube; and
providing a second catheter assembly, wherein the second catheter assembly comprises:
a second catheter adapter, comprising a distal end, a proximal end, a side port disposed between the distal end of the second catheter adapter and a proximal end of the second catheter adapter, and a lumen extending through the distal end of the second catheter adapter and the proximal end of the second catheter adapter;
a second catheter extending from the distal end of the second catheter adapter, wherein the second catheter comprises a second catheter length, wherein the first catheter length is less than the second catheter length;
a second extension tube extending from the side port of the second catheter adapter, wherein the second extension tube comprises a second extension tube length, wherein the first extension tube is longer than the second extension tube; and
a second connector coupled to a proximal end of the second extension tube, wherein the second connector comprises a second connector length,
wherein an entire length of the first catheter assembly from a distal tip of the first catheter to a proximal end of the first connector is equal to an entire length of the second catheter assembly from a distal tip of the second catheter to a proximal end of the second connector.

23. The method of claim 22, wherein the first connector comprises a first connector length, wherein the second connector comprises a second connector length, wherein the first connector length is equal to the second connector length.

24. A system for advancing a medical device, comprising:
a first catheter assembly comprising:
a first catheter adapter, comprising a distal end, a proximal end, and a lumen extending through the distal end and the proximal end;
a first catheter extending from the distal end of the first catheter adapter, wherein the first catheter comprises a first length; and
a first indicator marking corresponding to the first length; and/or
a second catheter assembly comprising:
a second catheter adapter, comprising a distal end, a proximal end, and a lumen extending through the distal end and the proximal end;
a second catheter extending from the distal end of the second catheter adapter, wherein the second catheter comprises a second length; and
a second indicator marking corresponding to the second length; and
an instrument advancement device configured to couple to the first catheter assembly and the second catheter assembly, wherein the instrument advancement device is configured to advance an instrument distally into the first catheter assembly and the second catheter assembly, wherein the instrument advancement device comprises a first other indicator marking matching the first indicator marking and a second other indicator marking matching the second indicator marking.

25. The system of claim 24, wherein the first indicator marking and the second indicator marking are arranged on the first and second catheter adapters, respectively.

26. The system of claim 24, wherein the first catheter assembly further comprises:
a first connector in fluid communication with the first catheter, the first connector having an inlet and at least two outlets;
a first needless access connector in fluid communication with one of the at least two outlets;
a first luer adapter in fluid communication with the other of the at least two outlets;
a first conduit connecting the first connector and the first luer adapter; and
a first clamp arranged on the first conduit,
wherein the second catheter assembly further comprises:
a second connector in fluid communication with the second catheter, the second connector having an inlet and at least two outlets;
a second needless access connector in fluid communication with one of the at least two outlets;
a second luer adapter in fluid communication with the other of the at least two outlets;
a second conduit connecting the second connector and the second luer adapter; and
a second clamp arranged on the second conduit.

27. The system of claim 26, wherein the first indicator marking and the second indicator marking are arranged on the first needless access connector and the second needless access connector, respectively.

28. The system of claim 26, wherein the first needless access connector comprises a first housing, a first cap at an end of the first housing, and a first piston received within the first housing, and
wherein the second needless access connector comprises a second housing, a second cap at an end of the second housing, and a second piston received within the second housing.

29. The system of claim 28, wherein the first indicator marking and the second indicator marking are arranged on the first housing, the first cap, and/or the first piston and the second housing, the second cap, and/or the second piston, respectively.

30. The system of claim 26, wherein the first indicator marking and the second indicator marking are arranged on the first luer adapter and the second luer adapter, respectively.

31. The system of claim 26, wherein the first indicator marking and the second indicator marking are arranged on the first clamp and the second clamp, respectively.

32. A kit comprising:
a catheter assembly comprising:
a catheter adapter, comprising a distal end, a proximal end, and a lumen extending through the distal end and the proximal end;
a catheter extending from the distal end of the catheter adapter, wherein the catheter comprises a length; and
an indicator marking corresponding to the length; and
a wound cover comprising:
a dressing;
a seal; and
another indicator marking matching the indicator marking.

33. The kit of claim 32, wherein the indicator marking, is arranged on the dressing.

34. The kit of claim 32, wherein the indicator marking is arranged on the seal.

35. The kit of claim 32, further comprising a label configured to be placed on the dressing and/or the seal, and wherein the indicator marking is arranged on the label.
